(19) United States
(12) Patent Application Publication
(10) Pub. No.: US 2024/0139333 A1
(43) Pub. Date: May 2, 2024

(54) 2,6-DICHLOROPHENYL ESTER COMPOUNDS AND USE THEREOF

(71) Applicant: Fusion Pharmaceuticals Inc., Hamilton (CA)

(72) Inventors: Matthew Guest, Carlsbad, CA (US); Michael B. Johansen, Cambridge (CA); Stuart J. Mahoney, Brantford (CA)

(21) Appl. No.: 18/512,575

(22) Filed: Nov. 17, 2023

Related U.S. Application Data
(63) Continuation of application No. PCT/US2022/031314, filed on May 27, 2022.
(60) Provisional application No. 63/194,008, filed on May 27, 2021.

(51) Int. Cl. A61K 47/68; A61K 47/60

(52) U.S. Cl. CPC A61K 47/6887; A61K 47/60; A61K 47/6849

(57) ABSTRACT

The present disclosure relates to 2,6-dichlorophenylester compounds. Also disclosed are methods for using the same for the preparation of certain conjugates.

### RELATED APPLICATIONS

This application is a continuation of International Application Number PCT/US2022/031314, filed on May 27, 2022, which claims priority to U.S. Provisional Patent Application No. 63/194,008, filed on May 27, 2021, the entire contents of each of which are hereby incorporated by reference for all purposes.

### BACKGROUND

Hydroxamic acid (HOA) and hydroxypyridinone (HOPO) are organic moieties capable of chelating a variety of metals to form metal complexes (see Chem. Rev. 2018, 118, 7657). For example, they can chelate with certain radiometals such as alpha- or beta-emitters to provide therapeutically useful radiopharmaceuticals when conjugated to targeting vectors including antibodies (aka radioimmunoconjugates).

Currently, the typical preparation of conjugates comprising HOA or HOPO moieties utilizes cross linkers such as maleimides to react with thiols (e.g., cysteine) or amines (e.g., lysine). Other cross linkers include aryl isothiocyanates and squaramides. These methods have drawbacks including low stability and poor yields, and can also introduce an increase in undesirable hydrophobicity to the resulting conjugate as a result of the incorporation of the cross linker moiety into the conjugate (not a traceless conjugation).

Alternative approaches that have been described utilize strategies of protecting the HOA or HOPO active moieties during the conjugation step. These approaches typically include protections as a metal complex (e.g., Fe chelation; see Org. Proc. Res. Dev. 2016, 20, 312), which currently necessitates a demetallation step prior to radiolabeling, or protection of the N-O nucleophilic moiety (e.g., 2-nitrobenzyl protection; see PCT Int. Appl. (2011), WO2011079291) which also requires a deprotection step that may subject the resulting conjugate to incompatible conditions during the deprotection and/or further complicate characterization to ensure structural integrity and homogeneity.

As a result of the nucleophilicity of the HOA or HOPO moieties the otherwise prevalent active ester strategy in bioconjugations has been notably absent without employing N-O protection strategies as mentioned above. Conversely, relatively inert esters such as ethylester have been reported as HOPO substituents in the literature for some time (see J. Am Chem. Soc. 1954, 76, 3168).

There is a need to develop new methods for preparing conjugates comprising HOA or HOPO moieties without the above-described drawbacks.

### SUMMARY

The present disclosure relates to 2,6-dichlorophenylester compounds that provide unexpected superiority for synthesizing conjugates comprising HOA or HOPO moieties without requiring protection and deprotection steps to enable bioconjugation.

The unique feature of preparing conjugates comprising HOA or HOPO moieties without protecting the active moieties is advantageous over the known methods in the field for synthesizing HOA- or HOPO-containing bifunctional chelates. The 2,6-dichlorophenylester (or its equivalent thioester) compounds disclosed in this invention provide the right balance of electrophilicity to enable increased stability with regards to degradation (including hydrolysis) while not sacrificing the ability to act as a tool for bioconjugation with amines (e.g., amino acid such as lysine) under mild conditions. In one aspect, the present invention relates to methods of preparing a conjugate of formula I:

A-L-C(═O)—B (I),

wherein A is H, heteroalkyl, 5-20 membered heterocycloalkyl, aryl, or heteroaryl, each of heteroalkyl, 5-20 membered heterocycloalkyl, aryl, and heteroaryl being optionally substituted; L is a linker selected from the group consisting of C₁₋₅₀ alkyl, C₁₋₅₀ heteroalkyl, C₃₋₂₀ cycloalkyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, heterocycloalkyl, aryl, and heteroaryl being optionally substituted; and B is a moiety comprising an amine unit,
wherein the method comprises: providing a compound of A-L-C(═O)-M-X, in which M is O or S and X is 2,6-dichlorophenyl; and conjugating the compound of A-L-C(═O)-M-X with a molecule comprising an amine unit to afford the conjugate of formula I.

In some embodiments, variable A is a heteroalkyl group comprising one or more hydroxamic acid units, a heteroalkyl group substituted directly or indirectly with one or more hydroxypyridinone units, or a 5-20 membered heterocycloalkyl group substituted directly or indirectly with one or more acetic acid or hydroxypyridinone units.

In some embodiments, variable L is optionally substituted CC₁₋₅₀ alkyl, optionally substituted C₁₋₅₀ heteroalkyl, C═O, —NH—(C═O)—, or a combination thereof, wherein the optionally substituted C₁₋₅₀ heteroalkyl comprises one or more oxygen atoms.

In another aspect, this invention relates to compounds having the structure of formula (II) below, or a metal complex thereof, or a salt thereof:

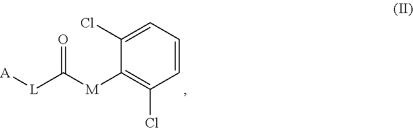

wherein A is C₁₋₅₀ heteroalkyl comprising one or more hydroxamic acid units, C₁₋₅₀ heteroalkyl substituted directly or indirectly with one or more hydroxypyridinone units, or 5-20 membered heterocycloalkyl substituted directly or indirectly with one or more acetic acid or hydroxypyridinone units; L is a linker selected from the group consisting of C₁₋₅₀ alkyl, C₁₋₅₀ heteroalkyl, C₃₋₂₀ cycloalkyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, heterocycloalkyl, aryl, and heteroaryl being optionally substituted; and M is O or S.

In some embodiments, the compounds described above comprise a metal complex that contains a metal selected from the group consisting of Bi, Pb, Y, Mn, Cr, Fe, Co, Zn, Ni, Tc, In, Ga, Cu, Re, a lanthanide, and an actinide.

In some embodiments, the compounds described above comprise a metal complex that contains a radionuclide selected from the group consisting of ⁸⁹Zr, ⁴⁷Sc, ⁵⁵Co, ⁶⁰Cu, ⁶¹Cu, C⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹In, ^{117m}Sn, ¹⁴⁹Pm, ⁵²Mn, ¹⁴⁹Tb , ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²⁷Ac, ²²³Ra and ²²⁷Th.

In some embodiments, the compounds described above comprise a radionuclide of ⁸⁹Zr, ¹¹¹In, or ²²⁵Ac.

### Definitions

As used herein, the term “alkyl” (including alkylene) refers to a saturated, linear or branched hydrocarbon moiety, such as methyl, methylene, ethyl, ethylene, propyl, propylene, butyl, butylene, pentyl, pentylene, hexyl, hexylene, heptyl, heptylene, octyl, octylene, nonyl, nonylene, decyl, decylene, undecyl, undecylene, dodecyl, dodecylene, tridecyl, tridecylene, tetradecyl, tetradecylene, pentadecyl, pentadecylene, hexadecyl, hexadecylene, heptadecyl, heptadecylene, octadecyl, octadecylene, nonadecyl, nonadecylene, icosyl, icosylene, triacontyl, and triacotylene. C₁₋₅₀ alkyl represents an alkyl group that comprises C₁₋₅₀ carbon atoms (e.g., C₃₋₆ alkyl comprising C₃₋₆ carbon atoms, C₃₋₈ alkyl comprising 3-8 carbon atoms, and C₆₋₁₂ alkyl comprising 6-12 carbon atoms). In various embodiments, said alkyl is a C₁₋₅₀ alkyl, C₁₋₃₀ alkyl or C₁₋₁₀ alkyl.

As used herein, the term “heteroalkyl” (including heteroalkylene) refers to an aliphatic moiety (e.g., alkyl or alkylene) containing at least one heteroatom selected from N, O, P, B, S, Si, Sb, Al, Sn, As, Se, and Ge. C₁₋₅₀ heteroalkyl represents a heteroalkyl group that comprises 1-50 carbon atoms (e.g., C₃₋₈ heteroalkyl comprising 3-8 carbon atoms, and C₆₋₁₂ heteroalkyl comprising 6-12 carbon atoms). In various embodiments, said heteroalkyl is a C₁₋₅₀ heteroalkyl, C₁₋₃₀ heteroalkyl or C₁₋₁₅ heteroalkyl. Examples of “heteroalkyl” include, but are not limited to, the following moieties:

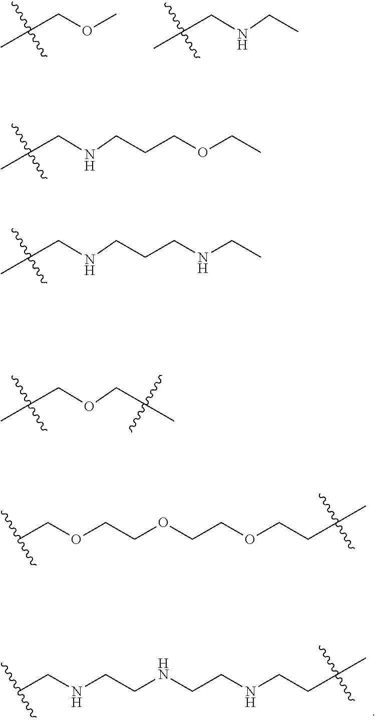

As used herein, the term “cycloalkyl” (including cycloalkylene) refers to a saturated hydrocarbon ring moiety, such as cyclopropyl, cyclobutyl, cyclopentyl, and cyclohexyl. C₃₋₂₀ cycloalkyl represents a cycloalkyl group that comprises 3-20 carbon atoms (e.g., C₃₋₈ cycloalkyl comprising 3-8 carbon atoms, and C₆₋₁₂ cycloalkyl comprising 6-12 carbon atoms). In various embodiments, said cycloalkyl is C₃₋₂₀ cycloalkyl, C₃₋₂₅ cycloalkyl or C₃₋₁₀ cycloalkyl.

As used herein, the term “heterocycloalkyl” (including heterocycloalkylene) refers to a cycloalkyl moiety (e.g., cycloalkyl or cycloalkylene) containing at least one heteroatom selected from N, O, P, B, S, Si, Sb, Al, Sn, As, Se, and Ge. Examples of “heterocycloalkyl” include, but are not limited to, a radical derived from aziridine, oxirane, azetidine, oxetane, pyrrolidine, tetrahydrofuran, tetrahydrothiophene, piperidine, piperazine, morpholine, or thiomorpholine. C₃₋₂₀ heterocycloalkyl represents a heterocycloalkyl group that comprises 3carbon atoms (e.g., C₃₋₈ heterocycloalkyl comprising 3-8 carbon atoms, and C₆₋₁₂ heterocycloalkyl comprising 6-12 carbon atoms).

As used herein, the term “5-20 membered heterocycloalkyl” represents an optionally substituted heterocycloalkyl ring that is 5-20 in size, i.e., comprising 5-20 atoms (e.g., C, N, O, and S). Examples of 5-20 membered (e.g., 9-18 membered) heterocycloalkyl include, but are not limited to, the following:

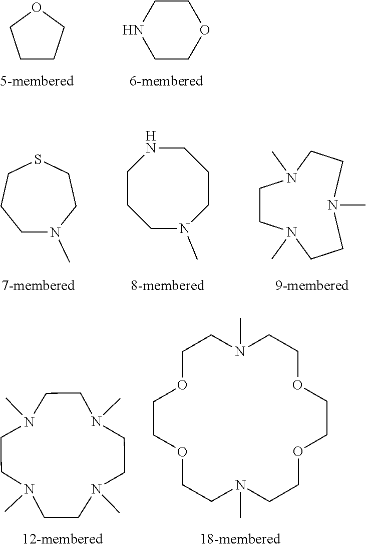

As used herein, the term “aryl” (including arylene) herein refers to a C₆ monocyclic, C₁₀ bicyclic, C₁₄ tricyclic, C₂₀ tetracyclic, or C₂₄ pentacyclic aromatic ring system. Examples of aryl groups include phenyl, phenylene, naphthyl, naphthylene, anthracenyl, anthracenylene, pyrenyl, and pyrenylene. In various embodiments, said aryl is a 5-6 membered aryl or a 5-8 membered aryl.

As used herein, the term “heteroaryl” (including heteroarylene) herein refers to an aromatic 5-8 membered monocyclic, 8-12 membered bicyclic, 11-14 membered tricyclic, and 15-20 membered tetracyclic ring system having one or more heteroatoms (such as O, N, S, or Se). Examples of heteroaryl groups include furyl, furylene, fluorenyl, fluorenylene, pyrrolyl, pyrrolylene, thienyl, thienylene, oxazolyl, oxazolylene, imidazolyl, imidazolylene, benzimidazolyl, benzimidazolylene, thiazolyl, thiazolylene, pyridyl, pyridylene, pyrimidinyl, pyrimidinylene, quinazolinyl, quinazolinylene, quinolinyl, quinolinylene, isoquinolyl, isoquinolylene, indolyl, and indolylene. In various embodiments, said heteroaryl is a 5-6 membered heteroaryl or a 5-8 membered heteroaryl.

Unless specified otherwise, alkyl, heteroalkyl, cycloalkyl, heterocycloalkyl, aryl, and heteroaryl mentioned herein include both substituted and unsubstituted moieties. Possible substituents on alkyl, heteroalkyl, cycloalkyl, heterocycloalkyl, aryl, and heteroaryl include, but are not limited to, C₁-C₁₀ alkyl, C₂-C₁₀ alkenyl, C₂-C₁₀ alkynyl, alkoxy, C₃-C₂₀ cycloalkyl, C₃-C₂₀ cycloalkenyl, C₃-C₂₀ heterocycloalkyl, C₃-C₂₀ heterocycloalkenyl, alkoxy, aryl, aryloxy, heteroaryl, heteroaryloxy, amino, C₁-C₁₀ alkylamino, C₂-C₂₀ dialkylamino, acylamino, diarylamino, alkylsulfonamino, arylsulfonamino, Ci-Cio alkylimino, arylimino, C₃-C₁₀ alkylsulfonimino, arylsulfonimino, hydroxyl, halo, oxo, thio, C₁-C₁₀ alkylthio, arylthio, Ci-Cio alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. Each of these groups or moieties refers to a substituent commonly used in the field and known to a skilled artisan. Further, cycloalkyl, cycloalkylene, cycloalkenyl, cycloalkenylene, heterocycloalkyl, heterocycloalkylene, heterocycloalkenyl, heterocycloalkenylene, aryl, and heteroaryl can also be fused with each other.

For example, C₁-C₅₀ heteroalkyl can be optionally substituted with one or more oxo (i.e., substitution of one or more CH₂ units with oxo to form C═O units), as exemplified below:

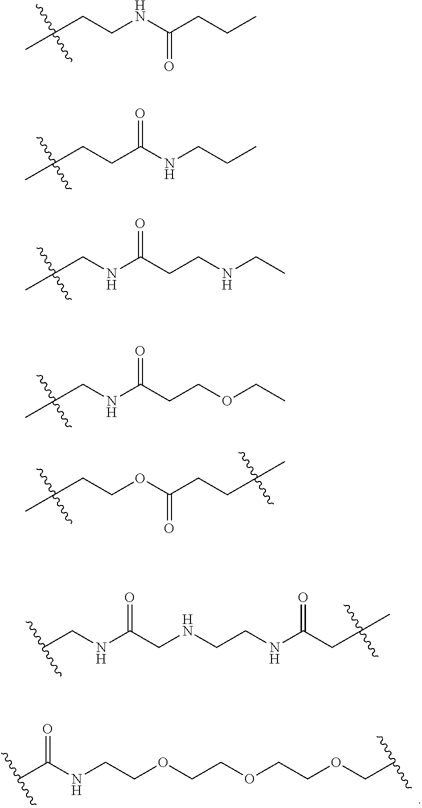

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or IgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains and may include sequence elements found in the same polypeptide chain or in different polypeptide chains.

As used herein, “antigen-binding fragment” refers to a portion of an antibody that retains the binding characteristics of the parent antibody.

The term “chelate” as used herein, refers to an organic compound or portion thereof that can be bonded to a central metal or radiometal atom at two or more points.

The term “conjugate,” as used herein, refers to a molecule that contains a chelating group or metal complex thereof, a linker group, and which optionally contains an antibody or antigen-binding fragment thereof.

As used herein, the term “compound,” is meant to include all stereoisomers, geometric isomers, and tautomers of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis.

As used herein, the term “radionuclide,” refers to an atom capable of undergoing radioactive decay (e.g. ⁸⁹Zr, ⁴⁷Sc, ⁵⁵Co, ⁶⁰Cu, ⁶¹Cu, C⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²⁷Ac, ²²³Ra and ²²⁷Th. The terms radioactive nuclide, radioisotope, or radioactive isotope may also be used to describe a radionuclide. Radionuclides may be used as detection agents, as described above. In some embodiments, the radionuclide may be an alpha-emitting radionuclide.

The term “radioimmunoconjugate,” as used herein, refers to any radioconjugate that comprises a radioactive molecule attached to an immune substance, such as a monoclonal antibody, that can bind to cancer cells. A radioimmunoconjugate can carry radiation directly and specifically to cancer cells, thereby killing cancer cells without harming normal cells. Radioimmunoconjugates may also be used with imaging to help find cancer cells in the body.

The compounds of the invention may have ionizable groups so as to be capable of preparation as salts (e.g., pharmaceutically acceptable salts). These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, among others. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

The term “polypeptide” as used herein refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

The details of one or more embodiments of the disclosure are set forth in the description below. Other features, objects, and advantages of the disclosure will be apparent from the below drawing, description, and from the claims.

### BRIEF DESCRIPTION OF THE DRAWING

FIG. 1. Stability study of Compounds A-E in pH 3 (Citric Acid/Na₂HPO₄ buffer) over 8 hours.

FIG. 2. Stability study of Compounds A-E in pH 5 (Citric Acid/Na₂HPO₄ buffer) over 8 hours.

FIG. 3. Stability study of Compounds A-E in pH 7 (Citric Acid/NaHPO4 buffer) over 8 hours.

FIG. 4. Stability study of Compounds A-E in pH 9 (Na₂CO₃ NaHCO₃ buffer) over 8 hours.

FIG. 5. Stability study of Compounds A-E at 20° C. in H₂ O over 8 hours.

FIG. 6. Stability study of Compounds A-E at 50° C. in H₂ O over 8 hours.

FIG. 7. Stability study of Compounds A-E in DMSO over 4 hours at 22±2° C.

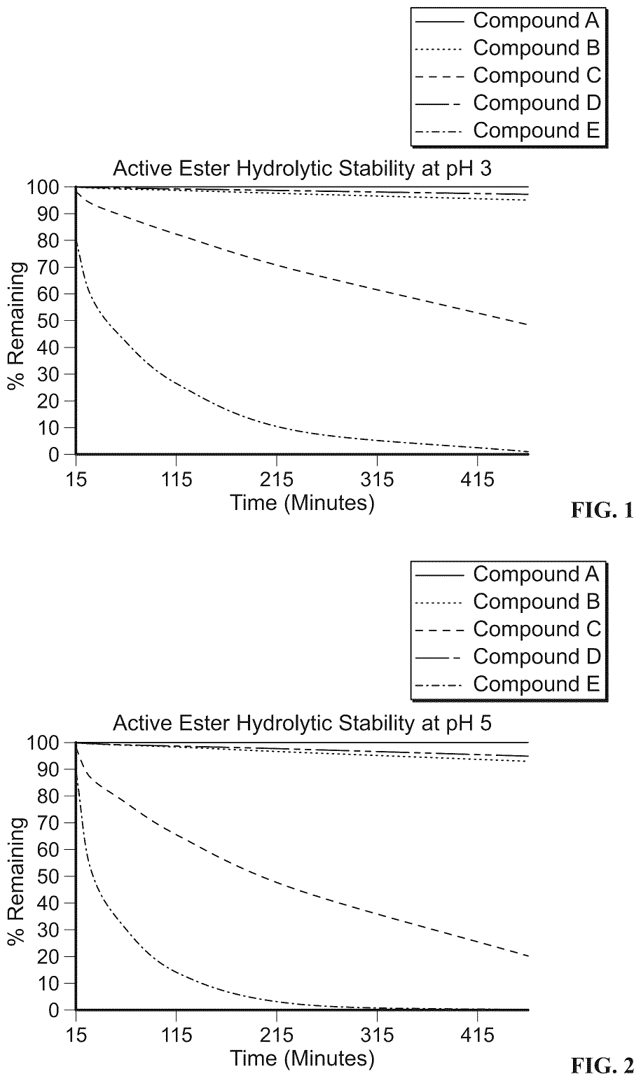

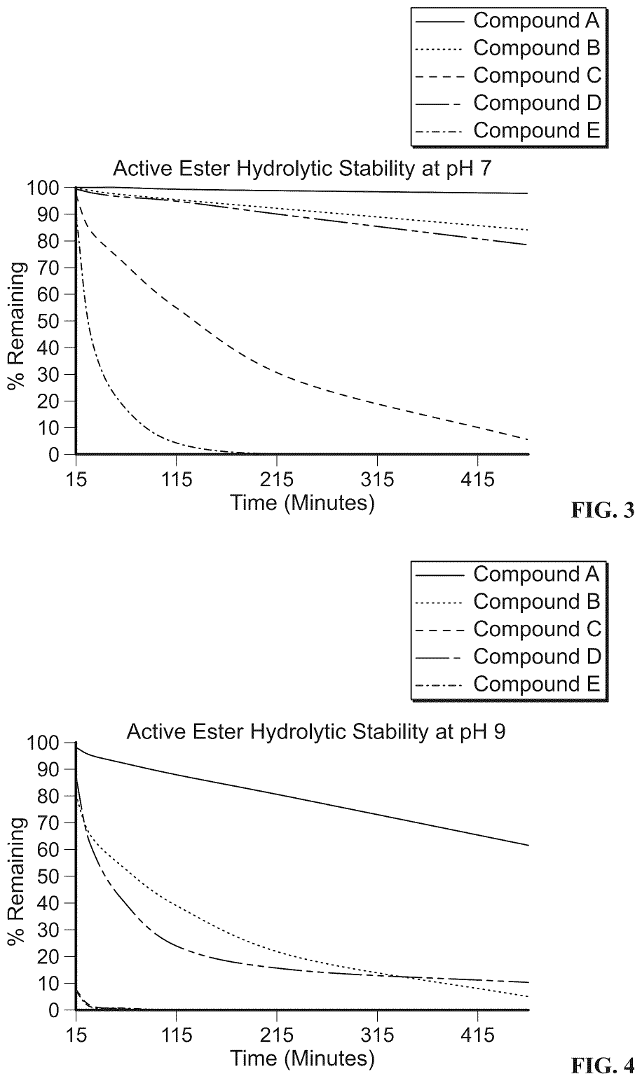

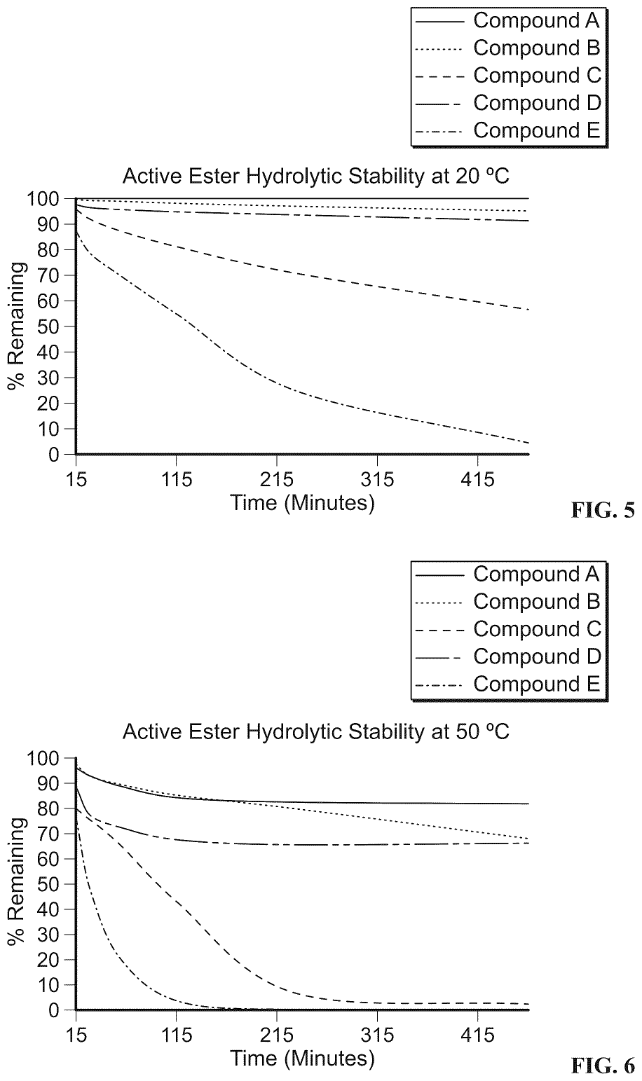

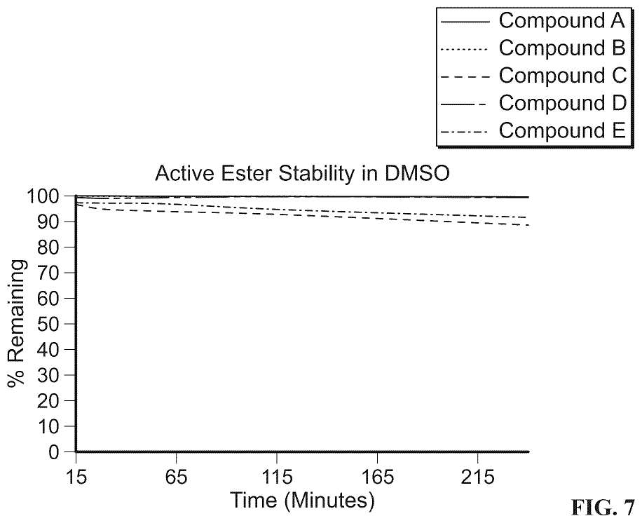

### DETAILED DESCRIPTION

The embodiments of the present disclosure relate to the structural identification of 2,6-dichlorophenyl ester compounds that have been found to have utility as cross linkers especially compounds containing hydroxypyridinone (HOPO) or hydroxamic acid (HOA) functionalities. The structural investigation also compared the reactivity profile of said 2,6-dichlorophenyl esters to commonly used active esters in the literature (e.g. N-hydroxy succinimide ester, p-nitrophenyl ester, 2,3,5,6-tetrafluorophenyl ester) as well as 2,6-difluorophenyl ester to demonstrate the superiority with respect to stability and functional group compatability while not sacrificing the ability to act as a lysine bioconjugation handle under mild conditions. Examples of the later include reactions of 2,6-dichlorophenylesters with lysine and a monoclonal antibody under mild conditions.

Notably HOPO and HOA functionalities are known to be prevalent and important donor moieties for metal scavengers and chelates. Herein disclosed are examples that demonstrate the synthesis of chelates bearing HOPO, HOA (exemplified by DFO) with the 2,6-dichlorophenyl ester moiety. In addition, a DOTA with carboxylic acid substitution such that typical active esters are highly prone to hydrolysis is described with the 2,6-dichlorophenyl ester moiety.

As provided in the SUMMARY section, the present invention provides in one aspect a unique method of preparing a conjugate of formula I:

A-L-C(—O)—B I,

wherein A is H, heteroalkyl, 5-20 membered heterocycloalkyl, aryl, or heteroaryl, each of heteroalkyl, 5-20 membered heterocycloalkyl, aryl, and heteroaryl being optionally substituted; L is a linker selected from the group consisting of C₁₋₅₀ alkyl, C₁₋₅₀ heteroalkyl, C₃₋₂₀ cycloalkyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, heterocycloalkyl, aryl, and heteroaryl being optionally substituted; and B is a moiety comprising an amine unit,
wherein the method comprises: (i) providing a compound of A-L-C(═O)—M—X, in which M is O or S and X is 2,6-dichlorophenyl; and (ii) conjugating the compound of A-L-C(═O)-M-X with a molecule comprising an amine unit to afford the conjugate of formula I.

Typically, variable B in formula I is a moiety formed from an amino acid (e.g., lysine), a peptide, an antibody, or an antigen-binding fragment of an antibody.

In some embodiments, variable B is an amine functionalized stationary phase conjugated with the compound of A-L-C(═O)-M-X to form the conjugate of A-L-C(═O)—B.

In some embodiments, variable B is an antibody, or an antigen-binding fragment thereof, that has a lysine unit conjugated with the compound of A-L-C(═O)-M-X to form the conjugate of A-L-C(═O)—B.

In some embodiments, variable A in formula I is a C₁₋₅₀ heteroalkyl.

In some embodiments, variable A in formula I is a heteroalkyl group comprising one or more hydroxamic acid units, a heteroalkyl group substituted directly or indirectly with one or more hydroxypyridinone units, or a 5-20 membered heterocycloalkyl group substituted directly or indirectly with one or more acetic acid or hydroxypyridinone units.

In some embodiments, variable A in formula I is a heteroalkyl group comprising one or more hydroxamic acid units, wherein the heteroalkyl group is optionally substituted with one or more oxo (=═O). For example, A has the structure shown below:

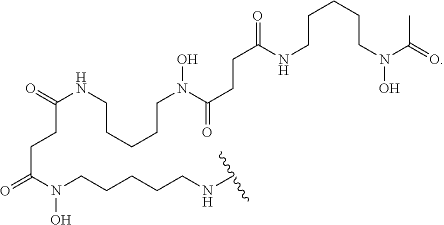

In some embodiments, variable A in formula I is a heteroalkyl group substituted directly or indirectly with one or more hydroxypyridinone units, wherein the heteroalkyl group comprises two or more nitrogen atoms. For example, A has the structure shown below:

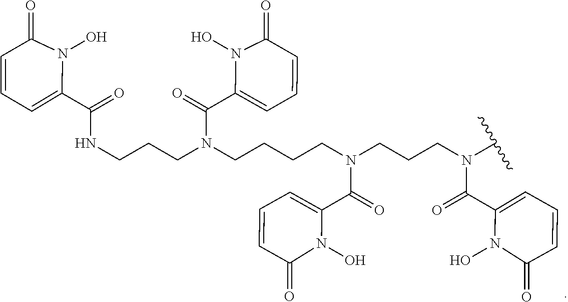 [SMILES: *N(CCCN(CCCCN(CCCNC(=O)c1cccc(=O)n1O)C(=O)c1cccc(=O)n1O)C(=O)c1cccc(=O)n1O)C(=O)c1cccc(=O)n1O]

In some embodiments, variable A in formula I is a 5-20 membered heterocycloalkyl group substituted directly or indirectly with one or more hydroxypyridinone units, wherein the 5-20 membered heterocycloalkyl group comprises three or more nitrogen atoms. For example, A has the structure shown below:

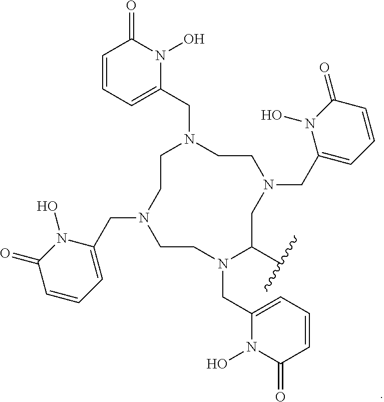 [SMILES: *C1CN(Cc2cccc(=O)n2O)CCN(Cc2cccc(=O)n2O)CCN(Cc2cccc(=O)n2O)CCN1Cc1cccc(=O)n1O]

In some embodiments, variable L in formula I is optionally substituted C₁₋₅₀ alkyl, optionally substituted C₁₋₅₀ heteroalkyl, C═O, —NH—(C═O)—, or a combination thereof, wherein the optionally substituted C₁₋₅₀ heteroalkyl comprises one or more oxygen atoms. In certain embodiments, L comprises 3-20 polyethylene glycol (PEG) units (e.g., 3 PEGs, 6 PEGs, 9 PEGs, 12 PEGs, or 15 PEGs).

In some embodiments, referring to formula I, variable A is a heteroalkyl group comprising one or more hydroxamic acid units, a heteroalkyl group substituted directly or indirectly with one or more hydroxypyridinone units, or a 5-20 membered heterocycloalkyl group substituted directly or indirectly with one or more acetic acid or hydroxypyridinone units; variable L is optionally substituted C1-50 alkyl, optionally substituted C₁₋₅₀ heteroalkyl, C═O, —NH—(C═O)—, or a combination thereof, wherein the optionally substituted C₁₋₅₀ heteroalkyl comprises one or more oxygen atoms; and variable B is an antibody, or an antigen-binding fragment thereof, that has a lysine unit conjugated with a compound of A-L-C(═O)—O—X (X being 2,6-dichlorophenyl) to form the conjugate of A-L-C(═O)—B.

In some embodiments, variable A in formula I is a 5-20 membered heterocycloalkyl group substituted directly or indirectly with one or more hydroxypyridinone units, wherein the 5-20 membered heterocycloalkyl group comprises three or more nitrogen atoms; variable

L in formula I is optionally substituted C₁₋₅₀ heteroalkyl, comprising one or more oxygen atoms; and variable B in formula I is an antibody, or an antigen-binding fragment thereof, that has a lysine unit conjugated with a compound of A-L-C(═O)—O—X (X being 2,6-dichlorophenyl) to form the conjugate of A-L-C(═O)—B.

In some embodiments, the method of this invention involves use of the compound of A-L-C(═O)-M-X (X being 2,6-dichlorophenyl) selected from one of the following:

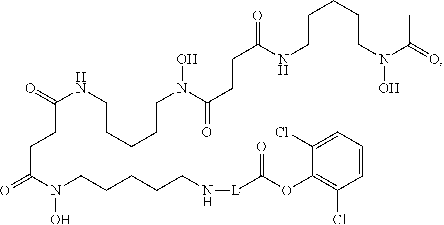

in which L is a linker selected from the group consisting of C₁₋₅₀ alkyl, C₁₋₅₀ heteroalkyl, C₃₋₂₀ cycloalkyl, C₄₋₂₀ cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted (e.g., substituted with oxo, hydroxy, cycloalkyl, or heterocycloalkyl).

In some embodiments, the method of this invention involves use of the compound of A-L-C(═O)-M-X (X being 2,6-dichlorophenyl) selected from one of the following:

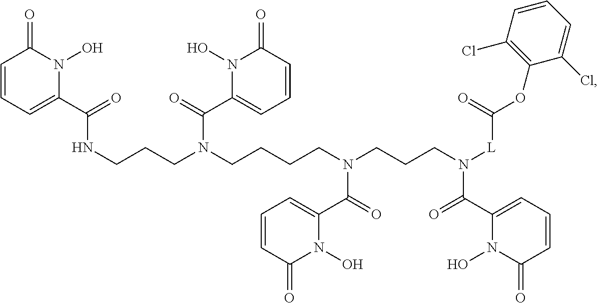

L being optionally substituted C₁₋₅₀ alkyl

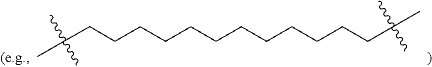 [SMILES: *CCCCCCCCCCCC*]

or optionally substituted C₁₋₅₀ heteroalkyl (e.g.,

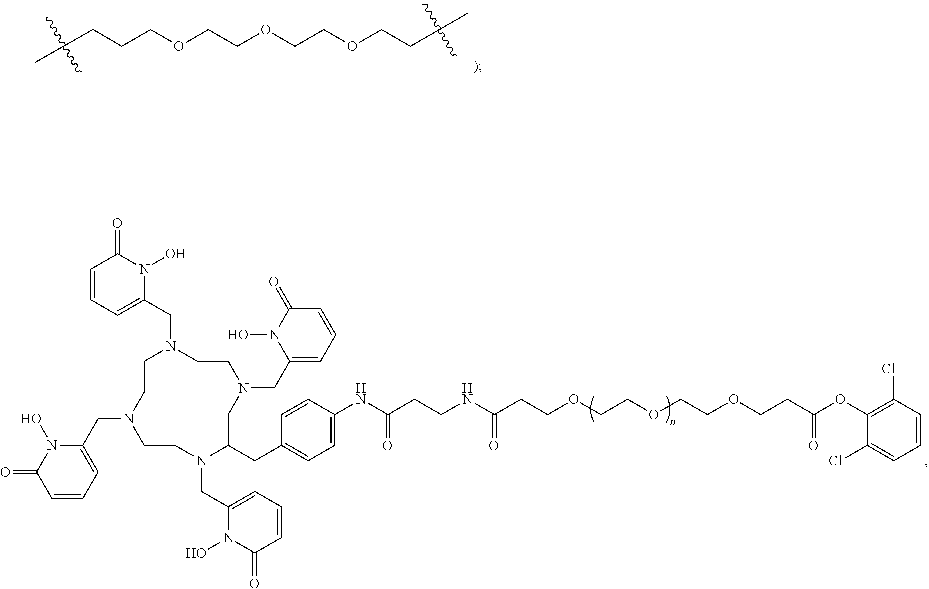

n being an integer of 1-10; and

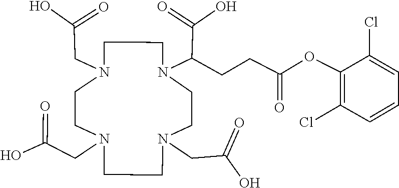 [SMILES: O=C(O)CN1CCN(CC(=O)O)CCN(C(CCC(=O)Oc2c(Cl)cccc2Cl)C(=O)O)CCN(CC(=O)O)CC1]

or a stereoisomer thereof (e.g., R-enantiomer).

In some embodiments, the method of this invention involves use of the compound of

A-L-C(═O)-M-X that has the following structure:

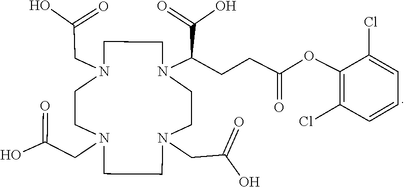 [SMILES: O=C(O)CN1CCN(CC(=O)O)CCN([C@H](CCC(=O)Oc2c(Cl)cccc2Cl)C(=O)O)CCN(CC(=O)O)CC1]

In some embodiments, the method of this invention involves use of the compound of A-L-C(═O)-M-X selected from one of the following:

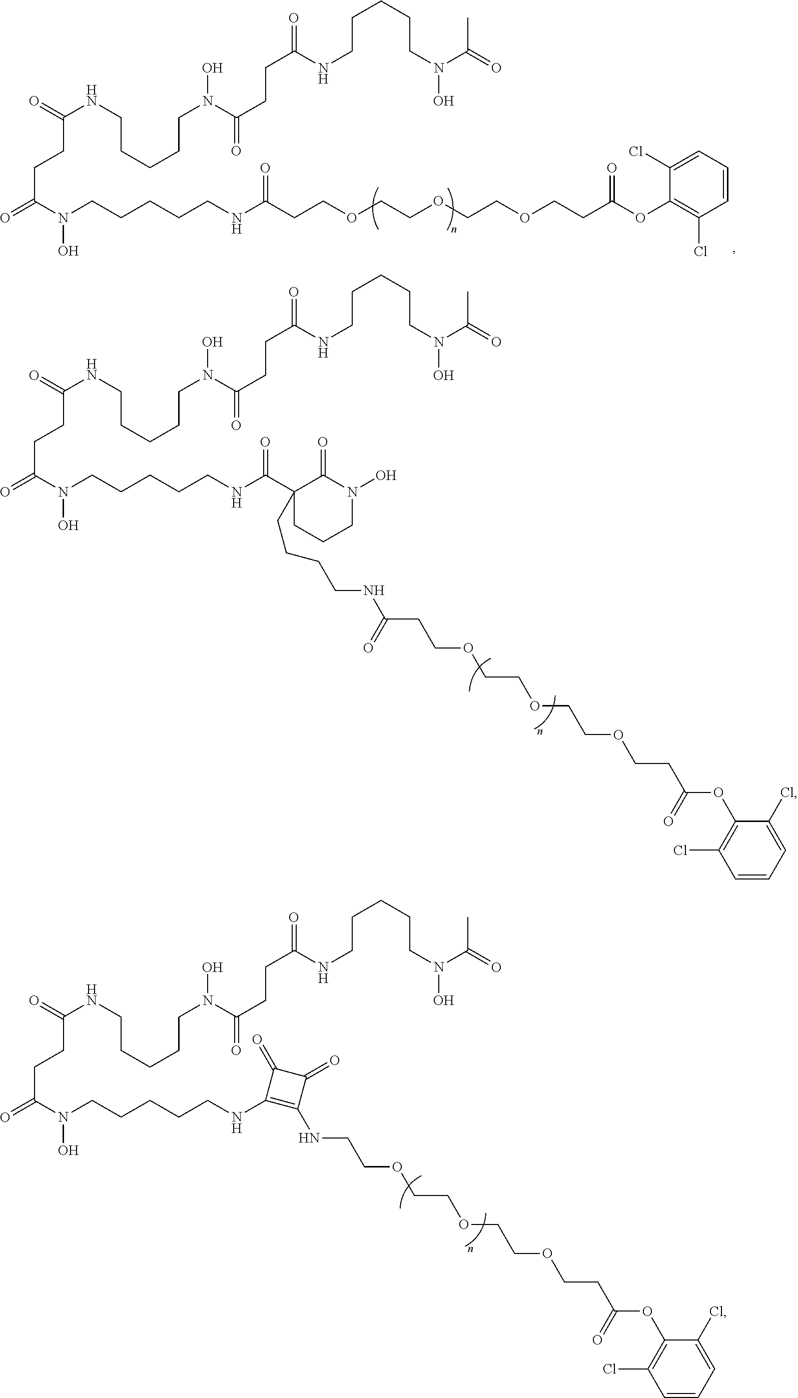

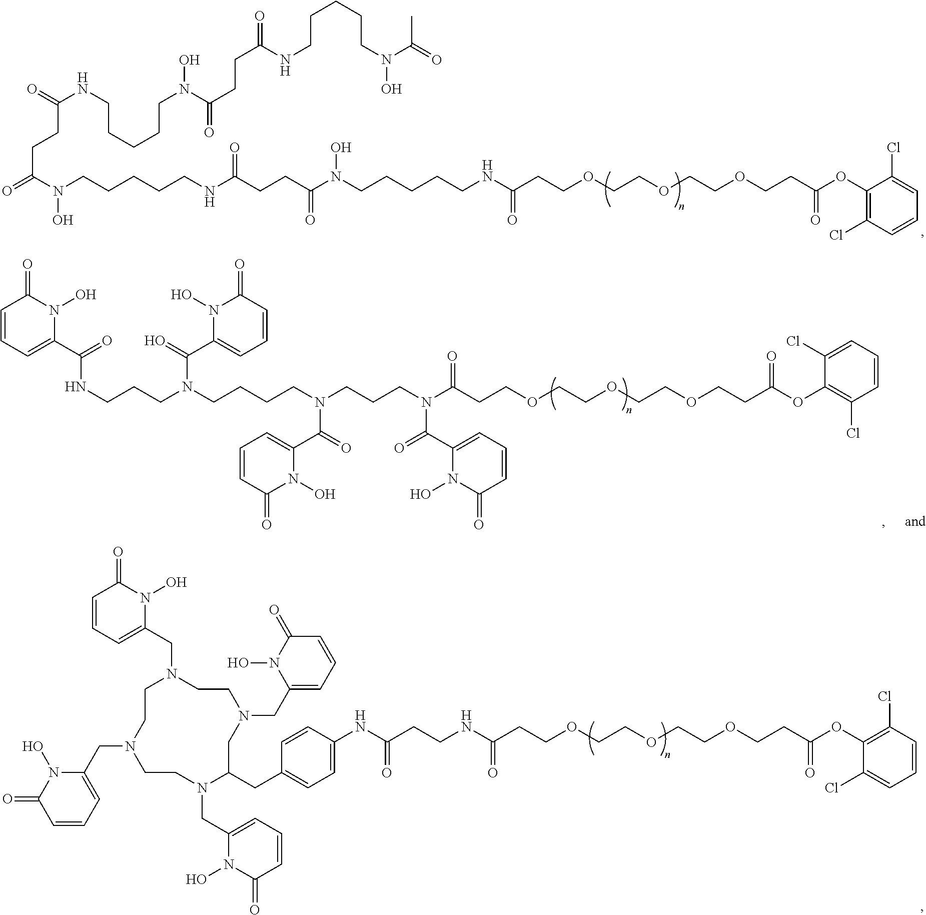

wherein each n, independently, is an integer of 1-10.

In some embodiments, the method of this invention includes the conjugation step that is performed at a temperature of 20-34° C. (e.g., about 21° C., about 22° C., about 23° C., about 24° C., about 25° C., about 26° C., about 27° C., about 28° C., about 29° C., about 30° C., about 31° C., about 32° C., about 33° C., or about 34° C.).

In some embodiments, the pH of the reaction mixture of the conjugation step is 5.0-10.0 (e.g., 5.0-6.0, 6.0-7.0, 7.0-8.0, 8.0-9.0, or 9.0-10.0). In certain embodiments, the pH of the reaction mixture of the conjugation step is less than 6.4 (e.g., about 6.3, about 6.2, about 6.1, about 6.0, about 5.9, about 5.8, about 5.7, about 5.6, about 5.5, about 5.4, about 5.2, about 5.1, or about 5.0).

As used herein, the term “about” refers to a ±10% variation from the recited quantitative value unless otherwise indicated or inferred from the context.

### Antibodies

Antibodies typically comprise two identical light polypeptide chains and two identical heavy polypeptide chains linked together by disulfide bonds. The first domain located at the amino terminus of each chain is variable in amino acid sequence, providing the antibody-binding specificities of each individual antibody. These are known as variable heavy (VH) and variable light (VL) regions. The other domains of each chain are relatively invariant in amino acid sequence and are known as constant heavy (CH) and constant light (CL) regions. Light chains typically comprise one variable region (VL) and one constant region (CL). An IgG heavy chain includes a variable region (VH), a first constant region (CH₁), a hinge region, a second constant region (CH2), and a third constant region (CH₃). In IgE and IgM antibodies, the heavy chain includes an additional constant region (CH4).

Antibodies described herein can include, for example, monoclonal antibodies, polyclonal antibodies, multispecific antibodies, human antibodies, humanized antibodies, camelid antibodies, chimeric antibodies, single-chain Fvs (scFv), disulfide-linked Fvs (dsFv), and anti-idiotypic (anti-Id) antibodies, and antigen-binding fragments of any of the above. In some embodiments, the antibody or antigen-binding fragment thereof is humanized. In some embodiments, the antibody or antigen-binding fragment thereof is chimeric. Antibodies can be of any type (e.g., IgG, IgE, IgM, IgD, IgA and IgY), class (e.g., IgG1, IgG2, IgG3, IgG4, IgA1 and IgA2) or subclass.

The term “antigen binding fragment” of an antibody, as used herein, refers to one or more fragments of an antibody that retain the ability to specifically bind to an antigen. Examples of binding fragments encompassed within the term “antigen binding fragment” of an antibody include a Fab fragment, a F(ab′)2 fragment, a Fd fragment, a Fv fragment, a scFv fragment, a dAb fragment (Ward et al., Nature, 1989, 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

In some embodiments, the antibody or antigen-binding fragment thereof disclosed in this invention is an insulin-like growth factor-1 receptor (IGF-1R) antibody or an antigen-binding fragment thereof. Examples of IGF-1R antibodies include figitumumab, cixutumumab, ganitumab, AVE1642 (also known as humanized EM164 and huEM164; see, e.g., U.S. Pat. No. 10,093,741), BIIB002, robatumumab, and teprotumumab.

In some embodiments, the antibody or antigen-binding fragment thereof disclosed in this invention is a fibroblast growth factor 3 (FGFR3) antibody or an antigen-binding fragment thereof. Non-limiting examples of FGFR3 antibodies include humanized monoclonal antibodies such as MFGR1877S (CAS No. 1312305-12-6; Genentech) (a human monoclonal antibody also known as vofatamab, and whose lyophilized form is also known as B-701 or R3Mab); PRO-001 (Prochon); PRO-007 (Fibron); IMC-Di i (Imclone); and AV-370 (Aveo Pharmaceuticals). (See, e.g., U.S. Pat. No. 8,410,250; U.S. Pat. No. 10,208,120; and International Patent Publication Nos. WO2002102972A2, WO2002102973A2, WO2007144893A2, WO2010002862A2, and WO2010048026A2).

In some embodiments, the antibody or antigen-binding fragment thereof disclosed in this invention is an endosialin (TEM-1) antibody or an antigen-binding fragment thereof. Non-limiting examples of TEM-1 antibodies include humanized monoclonal antibodies such as hMP-E-8.3 (Mediapharma), ontuxizumab (also known as MORAb-004) (Morphotek), and anti-TEM-1 antibodies from Kirin Brewery. (See, e.g., U.S. Pat. No. 8,895,000, and International Patent Publication Nos. WO 2017/134234 A1 and WO 2006/017759 A2.)

Antibodies or fragments described herein can be produced by any method known in the art for the synthesis of antibodies (see, e.g., Harlow et al., Antibodies: A Laboratory Manual, (Cold Spring Harbor Laboratory Press, 2nd ed. 1988); Brinkman et al., J. Immunol. Methods, 1995, 182:41-50; WO 92/22324; WO 98/46645). Chimeric antibodies can be produced using the methods described in, e.g., Morrison, Science, 1985, 229:1202, and humanized antibodies by methods described in, e.g., U.S. Pat. No. 6,180,370.

Additional antibodies described herein are bispecific antibodies and multivalent antibodies, as described in, e.g., Segal et al., J. Immunol. Methods, 2001, 248:1-6; and Tutt et al., J. Immunol., 1991, 147: 60, or any of the molecules described below.

In certain embodiments, the antibody or antigen-binding fragment thereof is a multi specific, e.g. bispecific. Multi specific antibodies (or antigen-binding fragments thereof) include monoclonal antibodies (or antigen-binding fragments thereof) that have binding specificities for at least two different sites.

In certain embodiments, amino acid sequence variants of antibodies or antigen-binding fragments thereof are contemplated; e.g., variants that bind to IGF-1R. For example, it may be desirable to improve the binding affinity and/or other biological properties of the antibody or antigen-binding fragment thereof. Amino acid sequence variants of an antibody or antigen-binding fragment thereof may be prepared by introducing appropriate modifications into the nucleotide sequence encoding the antibody or antigen-binding fragment thereof, or by peptide synthesis. Such modifications include, for example, deletions from and/or insertions into and/or substitutions of residues within the amino acid sequences of the antibody or antigen-binding fragment thereof. Any combination of deletion, insertion and substitution can be made to arrive at the final construct, provided that the final construct possesses desired characteristics, e.g. antigen binding.

Another aspect of this invention relates to compounds having the structure of formula (II) below, or a metal complex thereof, or a salt thereof:

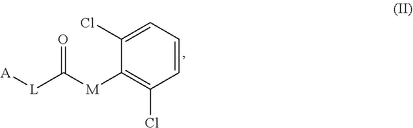

wherein A is C₁₋₅₀ heteroalkyl comprising one or more hydroxamic acid units, C₁₋₅₀ heteroalkyl substituted directly or indirectly with one or more hydroxypyridinone units, or 5-20 membered heterocycloalkyl substituted directly or indirectly with one or more acetic acid or hydroxypyridinone units; L is a linker selected from the group consisting of C₁₋₅₀ alkyl, C₁₋₅₀ heteroalkyl, C₃₋₂₀ cycloalkyl, C₄₋₂₀ cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted; and M is O or S.

In some embodiments, variable A in formula II is C₁₋₅₀ heteroalkyl (e.g., C₃₋₁₀ heteroalkyl, C₁₀₋₁₅ heteroalkyl, C₁₅₋₂₀ heteroalkyl, C₂₀₋₃₀ heteroalkyl, or C₃₀₋₄₀ heteroalkyl) comprising one or more hydroxamic acid units, wherein the heteroalkyl group is optionally substituted with one or more oxo (═O). For example, compounds of formula II can have A being the structure shown below:

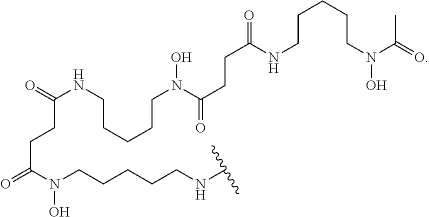

In some embodiment, variable A in formula II is C₁₋₅₀ heteroalkyl (e.g., C₃₋₁₀ heteroalkyl, C₁₀₋₁₅ heteroalkyl, C₁₅₋₂₀ heteroalkyl, C₂₀₋₃₀ heteroalkyl, or C₃₀₋₄₀ heteroalkyl) substituted directly or indirectly with one or more hydroxypyridinone units, wherein the heteroalkyl group comprises two or more nitrogen atoms. For example, compounds of formula II can have A being the structure shown below:

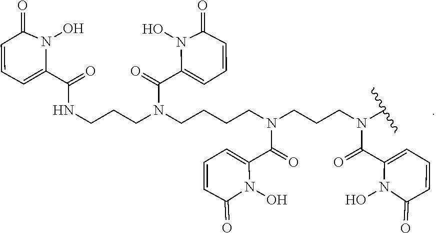 [SMILES: *N(CCCN(CCCCN(CCCNC(=O)c1cccc(=O)n1O)C(=O)c1cccc(=O)n1O)C(=O)c1cccc(=O)n1O)C(=O)c1cccc(=O)n1O]

In some embodiment, variable A in formula II is 5-20 membered heterocycloalkyl substituted directly or indirectly with one or more hydroxypyridinone units, wherein the 5-20 membered heterocycloalkyl group comprises three or more nitrogen atoms. For example, compounds of formula II can have A being the structure shown below:

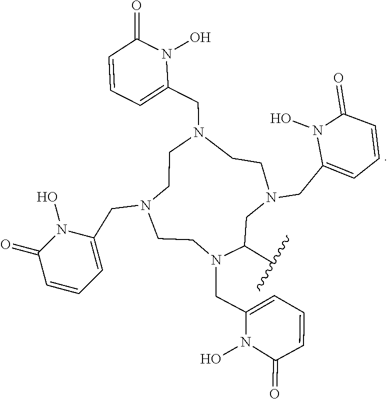 [SMILES: *C1CN(Cc2cccc(=O)n2O)CCN(Cc2cccc(=O)n2O)CCN(Cc2cccc(=O)n2O)CCN1Cc1cccc(=O)n1O]

In some embodiments, variable L in formula II is optionally substituted C1-50 alkyl, optionally substituted C₁₋₅₀ heteroalkyl, C═O, —NH—(C═O)—, or a combination thereof, wherein the optionally substituted C₁₋₅₀ heteroalkyl comprises one or more oxygen atoms. In certain embodiments, L comprises C₃₋₂₀ polyethylene glycol (PEG) units (e.g., 3 PEGs, 6 PEGs, 9 PEGs, 12 PEGs, or 15 PEGs).

In some embodiments, compounds of formula II have the structure shown below:

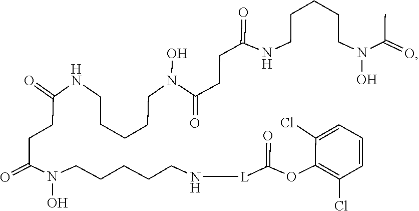

in which L is a linker selected from the group consisting of C₁₋₅₀ alkyl, C₁₋₅₀ heteroalkyl, C₃₋₂₀ cycloalkyl, C4-20 cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted (e.g., substituted with oxo, hydroxy, cycloalkyl, or heterocycloalkyl).

In some embodiments, compounds of formula II have the structure shown below:

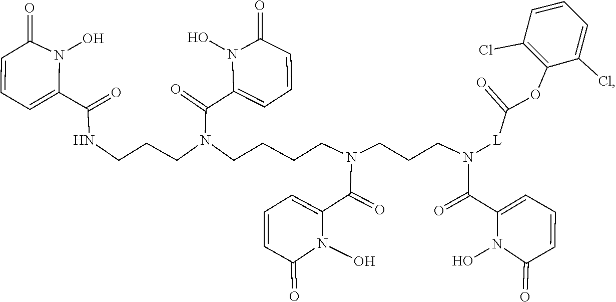

L being optionally substituted C₁₋₅₀ alkyl

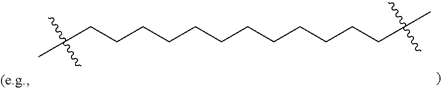 [SMILES: *CCCCCCCCCCCC*]

or optionally substituted C₁₋₅₀ heteroalkyl (e.g.,

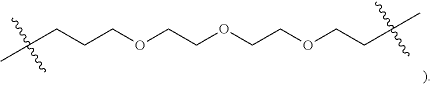 [SMILES: *CCCOCCOCCOCC*]

In some embodiments, compounds of formula II are selected from one of the following:

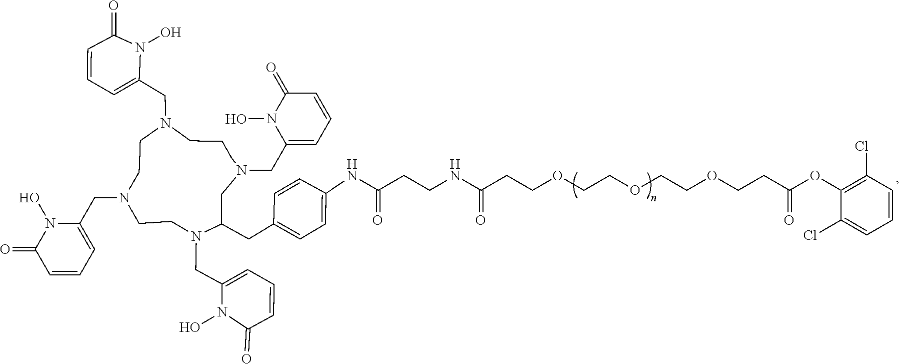

n being an integer of 1-10; and

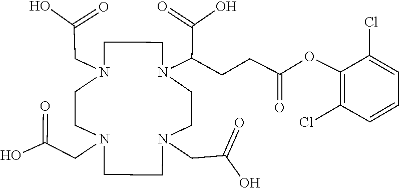 [SMILES: O=C(O)CN1CCN(CC(=O)O)CCN(C(CCC(=O)Oc2c(Cl)cccc2Cl)C(=O)O)CCN(CC(=O)O)CC1]

or a stereoisomer thereof (e.g., R-enantiomer).

In some embodiments, an exemplary compound of formula II has the following structure:

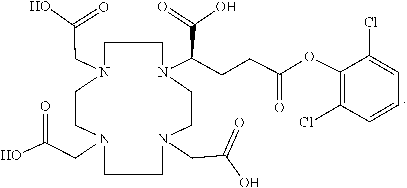 [SMILES: O=C(O)CN1CCN(CC(=O)O)CCN([C@H](CCC(=O)Oc2c(Cl)cccc2Cl)C(=O)O)CCN(CC(=O)O)CC1]

In some embodiments, compounds of formula II are selected from one of the following:

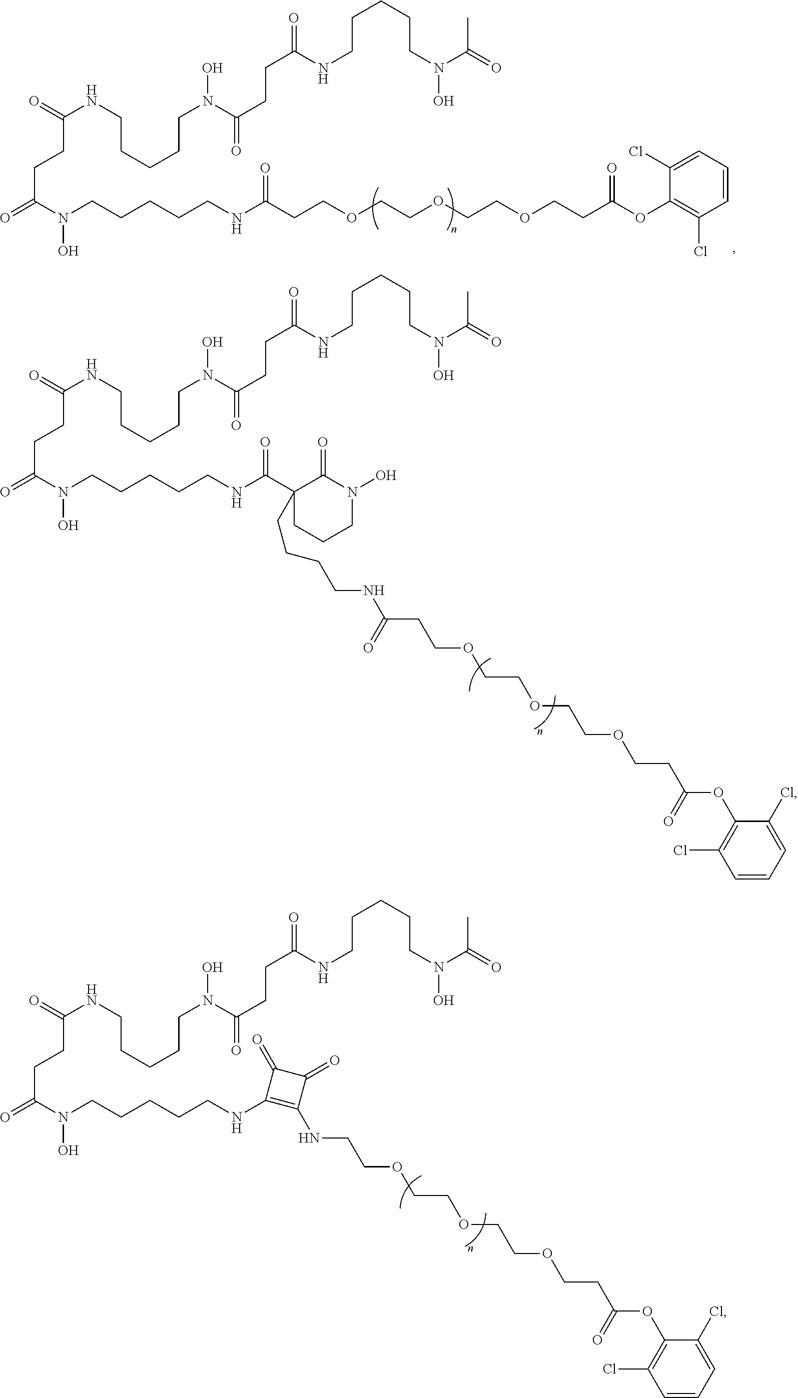

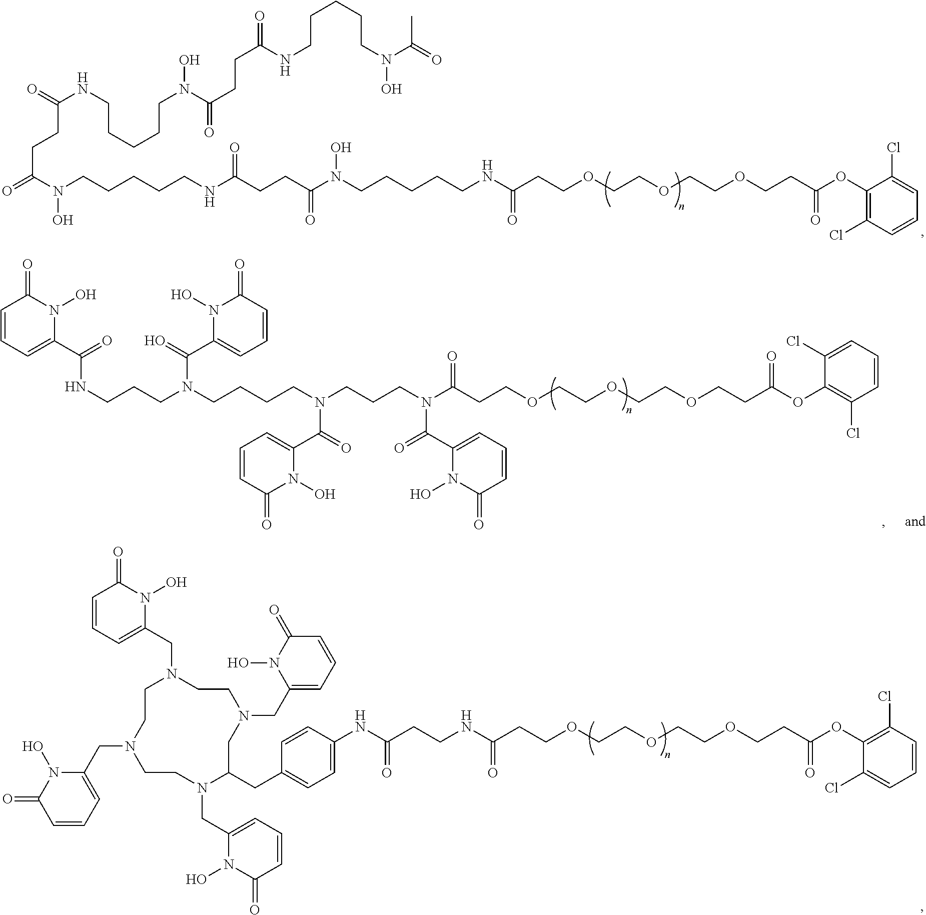

wherein each n, independently, is an integer of 1-10.

In some embodiments, compounds of formula II comprise a metal complex , wherein the metal of said metal complex is selected from the group consisting of Bi, Pb, Y, Mn, Cr, Fe, Co, Zn, Ni, Tc, In, Ga, Cu, Re, a lanthanide, and an actinide; or the metal of said metal complex is a radionuclide selected from the group consisting of ⁸⁹Zr, ⁴⁷Sc, ⁵⁵Co, ⁶⁰Cu, ⁶¹Cu, C⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²⁷Ac, ²²³Ra and ²²⁷Th.

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever.

### EXAMPLES

### Materials and General Methods

MALDI-TOF-MS (positive ion) was used to determine the chelate-to-antibody ratio of immunoconjugates. Matrix-Assisted Laser Desorption/Ionization Time-of-Flight Mass Spectrometry (MALDI-TOF-MS) was performed using a MALDI Bruker Ultraflextreme Spectrometer. A saturated solution of sinapinic acid was prepared in TA30 solvent (30:70 [v/v] acetonitrile: 0.1% TFA in water). The samples were mixed in a 1:1 ratio with the matrix solution. A sample volume of 1 μL was spotted on the plate and a protein solution of BSA was used as an external standard.

Size exclusion chromatography (SEC) was performed using a Waters system comprised of a Waters 1525 Binary HPLC pump, a Waters 2489 UV/Visible Detector (monitoring at 280 nm) and TOSOH TSKgel G3000SWxl, 7.8×300 mm column.

SEC HPLC Elution Method 1: The isocratic SEC method had a flow rate=1.0 mL/min, with a mobile phase of 0.022 M NaH₂PO₄, 0.047 M Na₂HPO₄, 0.60 M sodium chloride, 0.0038 M sodium azide, pH=7.

Analytical HPLC-MS was performed using a Waters Acquity HPLC-MS system comprised of a Waters Acquity Binary Solvent Manager, a Waters Acquity Sample Manager, a Water Acquity Column Manager (column temperature 30° C.), a Waters Acquity Photodiode Array Detector (monitoring at 254 nm and 214 nm), a Waters Acquity TQD with electrospray ionization and a Waters Acquity BEH C18, 2.1×50 mm (1.7 μ.m) column. Preparative HPLC was performed using a Waters HPLC system comprised of a Waters 1525 Binary HPLC pump, a Waters 2489 UV/Visible Detector (monitoring at 254 nm and 214 nm) and a Waters)(Bridge Prep C18 19 ×100 mm (5 μm) column or Waters XBridge Prep Phenyl 19 ×100 mm (5 μm).

HPLC elution method 1: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; wavelength=214, 254 nm; initial=98% A, 3 min=98% A, 8 min=75% A, 10 min=0% A, 11 min=98% A, 12 min=98% A.

HPLC elution method 2: Waters Acquity BEH C18 2.1 ×50 mm (1.7 μ.m) column; mobile phase A: H 2 O (0.1% v/v TFA); mobile phase B: acetonitrile (0.1% v/v TFA); flow rate=0.3 mL/min; wavelength=214, 254 nm; initial=90% A, 8 min=0% A, 10 min=0% A, 11 min=90% A, 12 min=90% A.

HPLC elution method 3: Waters Acquity BEH C18 2.1×50 mm (1.7 μm) column; mobile phase A: H₂O (0.1% v/v TFA); mobile phase B: acetonitrile (0.1% v/v TFA); flow rate=0.3 mL/min; wavelength=214, 254 nm; initial=95% A, 8 min=75% A, 10 min=0% A, 11 min=95% A, 12 min=95% A.

HPLC elution method 4: Waters Acquity HSS CN 2.1×50 mm (1.8 μ.m) column; mobile phase A: H₂ O (0.1% v/v TFA); mobile phase B: acetonitrile (0.1% v/v TFA); flow rate=0.3 mL/min; wavelength=214, 254 nm; initial=90% A, 8 min=0% A, 10 min=0% A, 11 min 32 90% A, 12 min 32 90% A.

### Example 1. Synthesis of 2,6-Dichlorophenyl-3-(pyridin-3-yl)propanoate (Compound A)

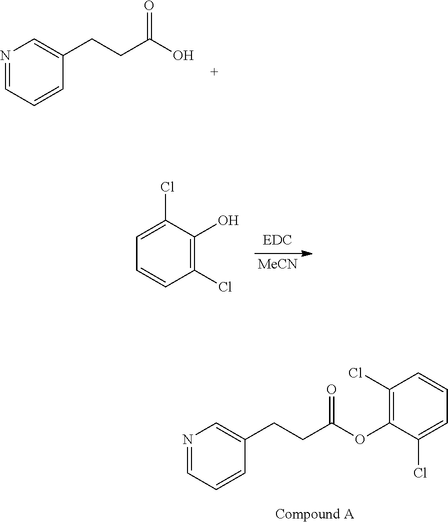 [SMILES: "MeCN" ; O=C(O)CCc1cccnc1 ; Oc1c(Cl)cccc1Cl ; O=C(CCc1cccnc1)Oc1c(Cl)cccc1Cl]

To a 100 mL round bottom flask containing 3-(pyridin-3-yl)propinoic acid (1.0 g, 6.62 mmol, 1 eq.) was added a stir bar followed by 50 mL of anhydrous acetonitrile. To this mixture was added 1-Ethyl-3-(3′-dimethylaminopropyl)carbodiimide (EDC, 1.13 g, 7.28 mmol, 1.1 eq.) in one portion followed by 2,6-dichlorophenol (1.19 g, 7.28 mmol, 1.1 eq.) in one portion. The reaction flask was capped and allowed to stir at room temperature (23° C.) for 4 hours while being monitored by HPLC-MS. The solution was then concentrated under reduced pressure and re-dissolved in dichloromethane (10 mL) and subsequently diluted with water (10 mL). The organic layer was extracted with dichloromethane (3×10 mL) and the combined organic extracts were dried over Na₂SO₄ and concentrated under reduced pressure.

The crude product was purified by flash column chromatography on silica (eluent: 1:1 hexanes to ethyl acetate) to afford Compound A (1.19 g, 60.7%, >99% purity) as a white solid. An aliquot was analyzed by HPLC-MS using elution method 2; retention time: 2.809 min; MS (positive ESI): found m/z 296.3 [M+H]⁺; C₁₄H₁₂Cl₂NO₂ (calc. 296.02). ¹H NMR (CDCl3, 600 MHz) 8.57 (d, J=1.8 Hz, 1H), 8.50 (d, J=4.5 Hz, 1H), 7.62 (app d, J=7.8 Hz, 1H), 7.35 (d, J=12.0 Hz, 2H), 7.26-7.24 (m overlapping with CHCl₃, 1H), 7.14 (t, J=6.0 Hz, 1H), 3.14 (t, J=7.7 Hz, 2H), 3.03 (t, J=7.6 Hz, 2H); 13 C UDEFT NMR (CDCl₃, 150 MHz) 168.84, 149.97, 148.11, 143.84, 135.91, 135.18, 128.83, 128.64, 127.25, 123.41, 34.65, 27.86.

### Example 2. Synthesis of 2,6-Difluorophenyl-3-(pyridin-3-yl)propanoate (Compound B)

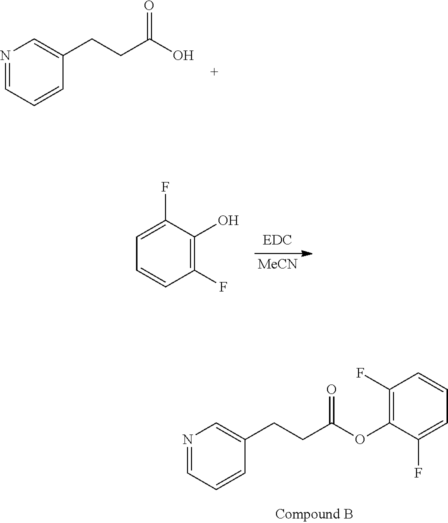 [SMILES: "MeCN" ; O=C(O)CCc1cccnc1 ; Oc1c(F)cccc1F ; O=C(CCc1cccnc1)Oc1c(F)cccc1F]

To a 100 mL round bottom flask containing 3-(pyridin-3-yl)propinoic acid (1.0 g, 6.62 mmol, 1 eq.) was added a stir bar followed by 50 mL of anhydrous Acetonitrile. To this mixture was added 1-Ethyl-3-(3′-dimethylaminopropyl)carbodiimide (EDC, 1.13 g, 7.28 mmol, 1.1 eq.) in one portion followed by 2,6-difluorophenol (0.86 g, 6.62 mmol, 1.0 eq.) in one portion. The reaction flask was capped and allowed to stir at room temperature (23° C.) for 4 hours while being monitored by HPLC-MS. The solution was then concentrated under reduced pressure and re-dissolved in dichloromethane (10 mL) and subsequently diluted with water (10 mL). The organic layer was extracted with dichloromethane (3×10 mL) and the combined organic extracts were dried over Na₂SO₄ and concentrated under reduced pressure. The crude product was purified by flash column chromatography on silica (eluent: 1:1 hexanes to ethyl acetate) to afford Compound B (1.06 g, 60.9%, 97% purity) as a white solid. An aliquot was analyzed by HPLC-MS using elution method 2; retention time: 2.348 min; MS (positive ESI): found m/z 264.7 [M+H]⁺; C₁₄H₁₂F₂NO₂ (calc. 264.08).

¹H NMR (CDCl₃, 600 MHz) 8.56 (d, J=6.0 Hz, 1H), 8.50 (dd, J=4.9, 1.3 Hz, 1H), 7.60 (dt, J=7.8, 2.3 Hz, 1H), 7.26-7.24 (m overlapping with CHCl₃, 1H), 7.19-7.15 (m, 1H), 6.99-6.95 (m, 2H), 3.11 (t, J=6.0 Hz, 2H), 2.99 (t, J=6.5 Hz, 2H); ¹³C UDEFT NMR (CDC₃, 150 MHz) 169.00, 155.16 (dd, J=249.0, 4.5 Hz), 149.91, 148.14, 135.82, 135.07, 127.07 (t, J=15.8 Hz), 126.46 (t, J=9.0 Hz), 123.45, 112.08 (dd, J=18.0, 4.5 Hz), 34.65, 27.96.

### Example 3. Synthesis of 2,3,5,6-Tetrafluorophenyl-3-(pyridin-3-yl)propanoate (Compound C)

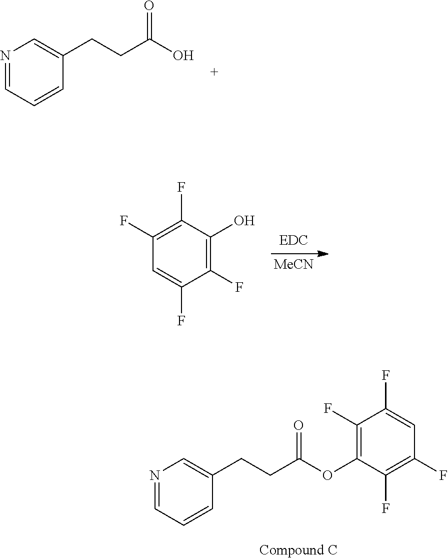 [SMILES: "MeCN" ; O=C(O)CCc1cccnc1 ; Oc1c(F)c(F)cc(F)c1F ; O=C(CCc1cccnc1)Oc1c(F)c(F)cc(F)c1F]

To a 100 mL round bottom flask containing 3-(pyridin-3-yl)propinoic acid (1.0 g, 6.62 mmol, 1 eq.) was added a stir bar followed by 40 mL of anhydrous acetonitrile. To this mixture was added 1-Ethyl-3-(3′-dimethylaminopropyl)carbodiimide (EDC, 1.13 g, 7.28 mmol, 1.1 eq.) in one portion. 2,3,5,6-Tetrafluorophenol (1.10 g, 6.62 mmol, 1.0 eq.) was then dissolved in 10 mL of anhydrous acetonitrile and added via syringe. The reaction flask was capped and allowed to stir at room temperature (23° C.) for 3 hours while being monitored by HPLC-MS. The solution was then concentrated under reduced pressure and re-dissolved in dichloromethane (10 mL) and subsequently diluted with water (10 mL). The organic layer was extracted with dichloromethane (3×10 mL) and the combined organic extracts were dried over Na₂SO₄ and concentrated under reduced pressure. The crude product was purified by flash column chromatography on silica (eluent: 1:1 hexanes to ethyl acetate) to afford Compound C (1.15 g, 59.0%, 95% purity) as a white solid. An aliquot was analyzed by HPLC-MS using elution method 2; retention time: 2.715 min; MS (positive ESI): found m/z 300.7 [M+H]⁺; C₁₄H₁₀F₄NO₂ (calc. 300.06). ¹H NMR (CDCl₃, 600 MHz) 8.56-8.51 (m, 2H), 7.61-7.59 (m, 1H), 7.28-7.26 (m overlapping with CHCl₃, 1H), 7.03-6.97 (m, 1H), 3.11 (t, J=9.0 Hz, 2H), 3.03 (t, J=9.0 Hz, 2H); ¹³ C UDEFT NMR (CDCl₃, 150 MHz) 168.26, 149.82, 148.27, 146.00 (dtd, J=246.7, 12.1 Hz, 5.0 Hz), 140.49 (ddd, J=248.2, 16.1, 6.2 Hz), 135.82, 134.72, 129.52 (br s), 123.52, 103.34 (t, J=22.5 Hz), 35.76, 27.07.

### Example 4. Synthesis of 4-Nitrophenyl-3-(pyridin-3-yl)propanoate (Compound D)

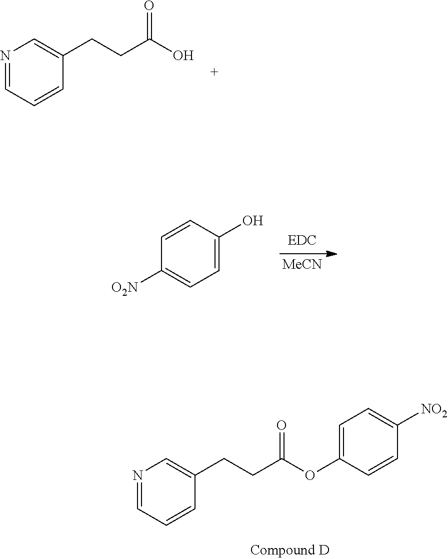 [SMILES: "MeCN" ; O=C(O)CCc1cccnc1 ; O=[N+]([O-])c1ccc(O)cc1 ; O=C(CCc1cccnc1)Oc1ccc([N+](=O)[O-])cc1]

To a 100 mL round bottom flask containing 3-(pyridin-3-yl)propinoic acid (1.0 g, 6.62 mmol, 1 eq.) was added a stir bar followed by 40 mL of anhydrous acetonitrile. To this mixture was added 1-Ethyl-3-(3′-dimethylaminopropyl)carbodiimide (EDC, 1.13 g, 7.28 mmol, 1.1 eq.) in one portion followed by 4-nitrophenol (0.92 g, 6.62 mmol, 1.0 eq.) in one portion. The reaction flask was capped and allowed to stir at room temperature (23° C.) for 24 hours while being monitored by HPLC-MS. The solution was then concentrated under reduced pressure and re-dissolved in dichloromethane (10 mL) and subsequently diluted with water (10 mL). The organic layer was extracted with dichloromethane (3×10 mL) and the combined organic extracts were dried over Na₂SO₄ and concentrated under reduced pressure. The crude product was purified by flash column chromatography on silica (eluent: 5:1 ethyl acetate to hexanes) to afford Compound D (1.06 g, 58.9%, 90% purity) as a white solid. An aliquot was analyzed by HPLC-MS using elution method 1; retention time: 8.078 min; MS (positive ESI): found m/z 272.9 [M+H]⁺; C₁₄H₁₃N₂O₄ (calc. 273.09). ¹H NMR (CDC; 3, 600 MHz) 8.56 (d, J=2.8 Hz, 1H), 8.52 (dd, J=5.3, 1.9 Hz, 1H), 8.27-8.25 (m, 2H), 7.60 (td, J =7.6, 2.0 Hz, 1H), 7.28-7.26 (m overlapping with CHCl₃, 1H), 7.22-7.20 (m, 2H), 3.09 (t, J =7.2 Hz, 2H), 2.96 (t, J=7.5 Hz, 2H); ¹³C UDEFT NMR (CDCl₃, 150 MHz) 169.95, 155.15, 149.89, 148.27, 145.40, 135.93, 135.02, 125.24, 123.52, 122.33, 35.43, 27.87.

### Example 5. Synthesis of 2,5-Dioxo-1-pyrrolidinyl 3-pyridinepropanoate (Compound E)

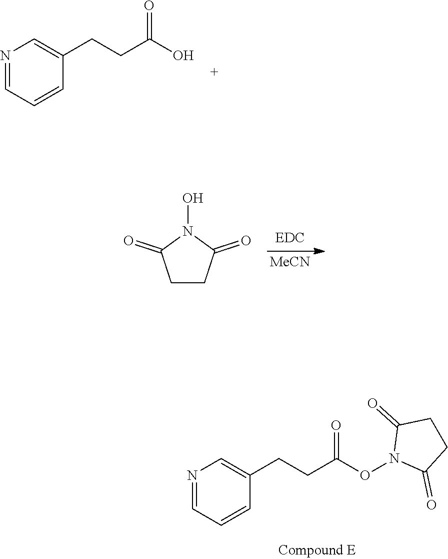 [SMILES: "MeCN" ; O=C(O)CCc1cccnc1 ; O=C1CCC(=O)N1O ; O=C(CCc1cccnc1)ON1C(=O)CCC1=O]

To a 100 mL round bottom flask containing 3-(pyridin-3-yl)propinoic acid (1.0 g, 6.62 mmol, 1 eq.) was added a stir bar followed by 40 mL of anhydrous acetonitrile. To this mixture was added 1-Ethyl-3-(3′-dimethylaminopropyl)carbodiimide (EDC, 1.13 g, 7.28 mmol, 1.1 eq.) in one portion followed by N-hydroxy succinimide (0.84 g, 6.62 mmol, 1.1 eq.) in one portion. The reaction flask was capped and allowed to stir at room temperature (23° C.) for 24 hours while being monitored by HPLC-MS. The solution was then concentrated under reduced pressure and re-dissolved in dichloromethane (10 mL) and subsequently diluted with water (10 mL). The organic layer was extracted with dichloromethane (3×10 mL) and the combined organic extracts were dried over Na₂SO₄ and concentrated under reduced pressure. The crude product was purified by flash column chromatography on silica (eluent: 5:1 ethyl acetate to hexanes) to afford Compound E (0.86 g, 52.1%, 90% purity) as a white solid. An aliquot was analyzed by HPLC-MS using elution method 1; retention time: 3.504 min; MS (positive ESI): found m/z 248.9 [M+H]⁺; C₁₂H₁₃N₂O₄ (calc. 249.09). ¹H NMR (CDCl₃, 600 MHz) 8.52-8.50 (m, 2H), 7.60 (td, J=7.8, 1.6 Hz, 1H), 7.28-7.26 (m overlapping with CHCl₃, 1H), 3.08 (t, J=7.9 Hz, 2H), 2.94 (t, J=7.7 Hz, 2H), 2.85 (br s, 4H); ¹³ C UDEFT NMR (CDC;₃, 150 MHz) 168.90, 167.47, 149.55, 148.03, 136.16, 134.58, 123.66, 32.20, 27.66, 25.56.

The following compounds were used for performing the studies described in Examples 1-10:

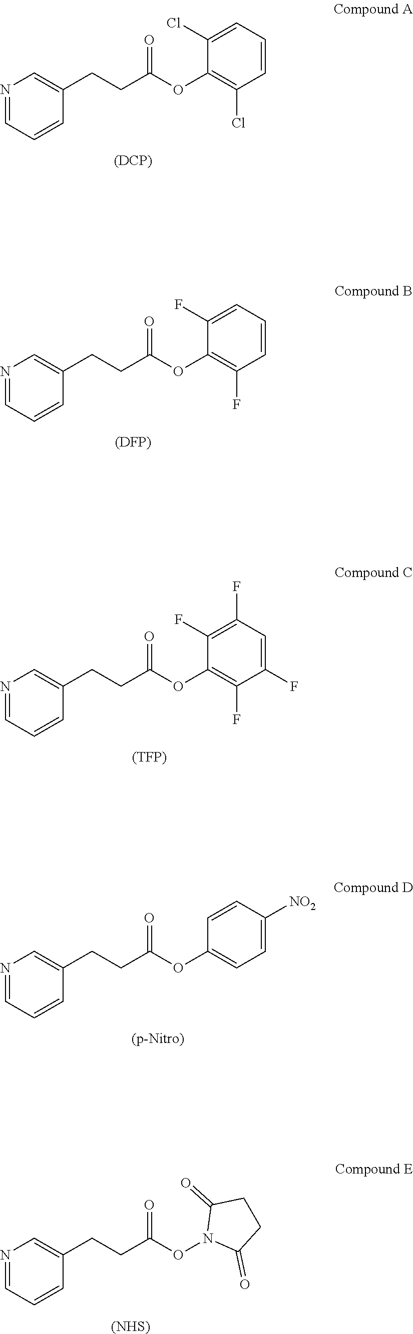

### Example 6. Functional Group Compatibility: Comparative Active Ester Reactivity Study with HOPO or HOA Nucleophiles

General Experimental Protocol for Comparative Active Ester Reactivity Study with HOPO or HOA Nucleophiles

To a 4 mL scintillation vial containing Compound A-E (in respective experiments; 1 eq.) was added a stir bar followed by 150 μL of HPLC grade acetonitrile. In another 4 mL scintillation vial containing a respective nucleophile (1 eq.) was added 280 μL HPLC grade water. The solutions were combined and allowed to stir at room temperature (22±2° C.) for 4 hours. Timed aliquots at time (T)=15, 30, 60, 120 and 240 minutes were taken and analyzed by HPLC-MS using elution method 1 (Table 1).

TABLE 1
Functional Group Compatibility: Comparative Active Ester Reactivity Study with HOPO or HOA Nucleophiles.

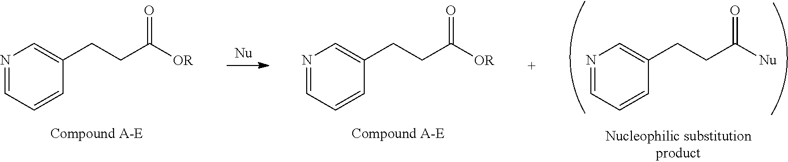

| Nucleophile | Timepoint (min.) | | | | |
|---|---|---|---|---|---|
| (1 eq.) | 15 | 30 | 60 | 120 | 240 |
| | Compound A (DCP) Reactions: | | | | |
| | % Compound A Remaining (% Nucleophilic Substitution Product) | | | | |
| 2- | 99.32 | 98.56 | 96.90 | 95.51 | 93.74 |
| Hydroxypyridine- | (0) | (0) | (0) | (0) | (0.26) |
| 1-oxide (1,2- | | | | | |
| HOPO) | | | | | |
| Benzhydroxamic | 97.05 | 95.97 | 92.36 | 86.65 | 78.07 |
| Acid | (2.95) | (4.03) | (7.64) | (13.35) | (21.93) |
| N-Hydroxy-N- | 99.57 | 99.24 | 98.36 | 96.11 | 92.22 |
| methylbenzamide | (0.43) | (0.66) | (1.53) | (3.77) | (7.62) |
| | Compound B (DFP) Reactions: | | | | |
| | % Compound B Remaining (% Nucleophilic Substitution Product) | | | | |
| 2- | 98.85 | 96.23 | 90.85 | 84.98 | 69.55 |
| Hydroxypyridine- | (0.33) | (0.54) | (0.98) | (2.05) | (1.67) |
| 1-oxide (1,2- | | | | | |
| HOPO) | | | | | |
| Benzhydroxamic | 81.80 | 69.62 | 56.57 | 42.81 | 28.60 |
| Acid | (18.10) | (30.25) | (43.26) | (56.93) | (70.96) |
| N-Hydroxy-N- | 91.32 | 84.96 | 73.90 | 56.72 | 35.97 |
| methylbenzamide | (8.58) | (14.88) | (25.92) | (43.06) | (63.71) |
| | Compound C (TFP) Reactions: | | | | |
| | % Compound C Remaining (% Nucleophilic Substitution Product) | | | | |
| 2- | 40.02 | 17.81 | 9.78 | 5.19 | 0.94 |
| Hydroxypyridine- | (10.07) | (12.74) | (11.09) | (4.07) | (1.70) |
| 1-oxide (1,2- | | | | | |
| HOPO) | | | | | |
| Benzhydroxamic | 58.86 | 45.60 | 32.39 | 18.88 | 9.26 |
| Acid | (40.72) | (53.89) | (67.00) | (80.24) | (89.53) |
| N-Hydroxy-N- | 81.12 | 72.43 | 61.68 | 50.09 | 37.80 |
| methylbenzamide | (18.44) | (27.06) | (37.69) | (49.10) | (61.03) |
| | Compound D (p-Nitro) Reactions: | | | | |
| | % Compound D Remaining (% Nucleophilic Substitution Product) | | | | |
| 2- | 99.18 | 96.81 | 93.29 | 85.25 | 75.07 |
| Hydroxypyridine- | (0.19) | (0.41) | (0.78) | (1.07) | (0.68) |
| 1-oxide (1,2- | | | | | |
| HOPO) | | | | | |
| Benzhydroxamic | 96.49 | 84.28 | 74.58 | 57.07 | 34.21 |
| Acid | (3.51) | (15.72) | (25.42) | (42.66) | (65.36) |
| N-Hydroxy-N- | 95.63 | 90.98 | 83.40 | 65.46 | 41.73 |
| methylbenzamide | (4.22) | (8.89) | (16.40) | (34.28) | (57.91) |
| | Compound E (NHS) Reactions: | | | | |
| | % Compound E Remaining (% Nucleophilic Substitution Product) | | | | |
| 2- | 15.47 | 12.28 | 11.06 | 7.15 | 3.69 |
| Hydroxypyridine- | (9.37) | (9.21) | (9.27) | (5.79) | (2.95) |
| 1-oxide (1,2- | | | | | |
| HOPO) | | | | | |
| Benzhydroxamic | 1.13 | 0.85 | 0.67 | 0.49 | 0.35 |
| Acid | (92.96) | (94.85) | (95.25) | (95.31) | (95.08) |
| N-Hydroxy-N- | 32.07 | 20.77 | 11.58 | 6.52 | 4.21 |
| methylbenzamide | (67.92) | (79.23) | (88.41) | (92.23) | (95.37) |

Note:
% Product Formation and % Active Ester Remaining is in comparison to any hydrolysis products observed in the 254 nm spectrum.

### Example 7. Nucleophilic Reactivity Study with L-Lysine.

General Experimental Protocol for Nucleophilic Reactivity Study with L-Lysine

To a 4 mL scintillation vial containing Compound A-E (in respective experiments; 1 eq.) was added a stir bar followed by 70 μL of HPLC grade acetonitrile. In another 4 mL scintillation vial containing L-Lysine monohydrochloride (5 eq.) was added 130 μL HPLC grade water followed by diisopropylethylamine (5 eq.). The solutions were combined and allowed to stir at room temperature (22±2° C.). Timed aliquots at time (T)=15, 30, 60 and 120 minutes were taken and analyzed by HPLC-MS using elution method 1 (Table 2).

TABLE 2
Nucleophilic Reactivity Study with L-Lysine.

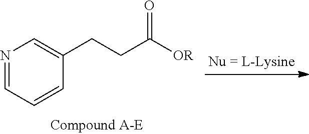

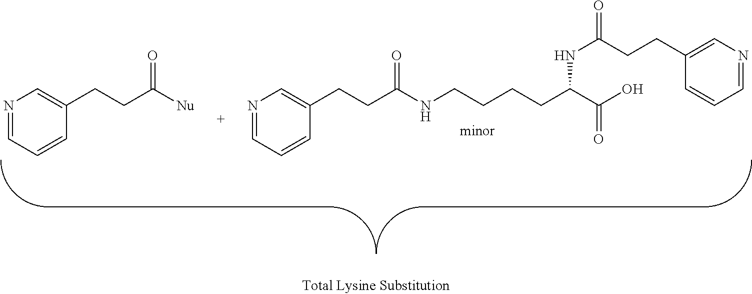

| | Active Esters (Compounds A-E) Reactions with L-Lysine (5 equiv): | | | |
|---|---|---|---|---|
| | % Total Lysine Substitution (% Active Ester Remaining) | | | |
| | Timepoint (min.) | | | |
| Active Ester | 15 | 30 | 60 | 120 |
| Compound A | 39.56 (60.44) | 73.69 (26.31) | 99.02 (0.98) | 100 (0) |
| Compound A^{a} | 99.14 (0.86) | 100 (0) | | |
| Compound B | 99.80 (0.20) | 100 (0) | | |
| Compound C | 100 (0) | | | |
| Compound D | 91.66 (8.34) | 100 (0) | | |
| Compound E | 100 (0) | | | |

Note:
% Total Lysine Substitution and % Active Ester Remaining is in comparison to any hydrolysis products observed in the 254 nm spectrum.
ª4-Dimethylaminopyridine (DMAP, 10 mol %) was added.

### Example 8: pH Stability of Compounds A-E

### General Experimental Protocol for pH Stability Study

To a 4 mL scintillation vial containing Compound A-E (in respective experiments; 0.8 mg) was added 4 mL of either a pH 3, pH 5 pH 7 or pH 9 buffer to create a 0.2 mg/mL solution. 2 mL of this solution was added to a 2 mL HPLC vial and placed in the auto sampler of an HPLC-MS at 20° C. Timed aliquots at time (T)=15, 30, 60, 120, 240 and 480 minutes were taken and analyzed by HPLC-MS using elution method 1 (Table 3).

pH 3 Buffer: Citric acid/Na₂HPO₄ (79.45 mL 0.1 M-citric acid and 20.55 mL 0.2-Na₂HPO₄)

pH 5 Buffer: Citric acid/Na₂HPO₄ (48.50 mL 0.1 M-citric acid and 51.50 mL 0.2-Na₂HPO₄)

pH 7 Buffer: Citric acid/Na₂HPO₄ (17.65 mL 0.1 M-citric acid and 82.35 mL 0.2-Na₂HPO₄)

pH 9 Buffer: Sodium Carbonate/Sodium Bicarbonate (10 mL 0.1 M-Na₂CO₃ and 90 mL 0.1-NaHCO₃)

TABLE 3
pH Stability of Compounds A-E.

| | Compound A (DCP) % Remaining | | | | | |
|---|---|---|---|---|---|---|
| | Timepoint (min.) | | | | | |
| pH | 15 | 30 | 60 | 120 | 240 | 480 |
| 3 | 100 | 100 | 100 | 100 | 100 | 100 |
| 5 | 100 | 100 | 100 | 100 | 100 | 100 |
| 7 | 100 | 100 | 100 | 99.35 | 98.75 | 97.82 |
| 9 | 98.15 | 95.27 | 92.61 | 87.81 | 79.28 | 61.56 |

Compound B (DFP) % Remaining
Timepoint (min.)

| pH | 15 | 30 | 60 | 120 | 240 | 480 |
|---|---|---|---|---|---|---|
| 3 | 100 | 99.56 | 99.19 | 98.69 | 97.41 | 95.05 |
| 5 | 100 | 99.50 | 99.05 | 98.27 | 96.36 | 92.97 |
| 7 | 99.35 | 98.45 | 97.44 | 95.40 | 91.66 | 84.15 |
| 9 | 78.39 | 65.31 | 54.47 | 38.67 | 19.98 | 5.01 |

Compound C (TFP) % Remaining
Timepoint (min.)

| pH | 15 | 30 | 60 | 120 | 240 | 480 |
|---|---|---|---|---|---|---|
| 3 | 98.19 | 93.92 | 89.61 | 82.13 | 68.98 | 48.46 |
| 5 | 96.06 | 86.91 | 79.07 | 65.20 | 45.26 | 20.20 |
| 7 | 94.61 | 83.82 | 73.25 | 54.53 | 27.99 | 5.60 |
| 9 | 7.60 | 1.71 | 0.49 | 0 | | |

Compound D (p-Nitro) % Remaining
Timepoint (min.)

| pH | 15 | 30 | 60 | 120 | 240 | 480 |
|---|---|---|---|---|---|---|
| 3 | 100 | 99.74 | 99.57 | 99.21 | 98.55 | 97.20 |
| 5 | 99.83 | 99.54 | 99.19 | 98.63 | 97.52 | 94.89 |
| 7 | 99.11 | 98.00 | 96.59 | 94.83 | 89.25 | 78.56 |
| 9 | 83.05 | 61.77 | 42.40 | 23.66 | 14.96 | 10.31 |

Compound E (NHS) % Remaining
Timepoint (min.)

| pH | 15 | 30 | 60 | 120 | 240 | 480 |
|---|---|---|---|---|---|---|
| 3 | 80.40 | 59.68 | 44.51 | 26.16 | 8.98 | 1.00 |
| 5 | 85.59 | 52.98 | 33.12 | 13.81 | 2.33 | 0 |
| 7 | 86.24 | 44.91 | 19.80 | 4.02 | 0 | |
| 9 | 6.98 | 1.20 | 0.74 | 0 | | |

Note:
% Active Ester Remaining is in comparison to any hydrolysis products observed in the 254 nm spectrum.

### Example 9: Temperature Stability of Compounds A-E in H2O.

### General Experimental Protocol for Temperature Stability Study

20° C.: To a 4 mL scintillation vial containing Compound A-E (in respective experiments; 0.8 mg) was added 4 mL of HPLC grade water to create a 0.2 mg/mL solution. 2 mL of this solution was added to a 2 mL HPLC vial and placed in the auto sampler of an HPLC-MS at 20° C. Timed aliquots at time (T)=15, 30, 60, 120, 240 and 480 minutes were taken and analyzed by HPLC-MS using elution method 1 (Table 4).

50° C.: To a 4 mL scintillation vial containing Compound A-E (in respective experiments; 0.8 mg) was added a stir bar followed by 4 mL of HPLC grade water to create a 0.2 mg/mL solution. The vial was capped and allowed to stir in a 50° C. oil bath for 8 hours. Timed aliquots at time (T) =15, 30, 60, 120, 240 and 480 minutes were taken and analyzed by HPLC-MS using elution method 1 (Table 4).

TABLE 4
Temperature Stability of Compounds A-E in H₂O.

| | Compound A (DCP) % Remaining | | | | | |
|---|---|---|---|---|---|---|
| | Timepoint (min.) | | | | | |
| Temperature (° C.) | 15 | 30 | 60 | 120 | 240 | 480 |
| 20 | 100 | 100 | 100 | 100 | 100 | 100 |
| 50 | 96.12 | 92.86 | 88.94 | 84.24 | 82.50 | 81.87 |
| | Compound B (DFP) % Remaining | | | | | |
| | Timepoint (min.) | | | | | |
| Temperature (° C.) | 15 | 30 | 60 | 120 | 240 | 480 |
| 20 | 99.63 | 99.20 | 98.92 | 98.12 | 96.98 | 95.14 |
| 50 | 97.57 | 92.71 | 89.62 | 85.20 | 79.93 | 68.06 |
| | Compound C (TFP) % Remaining | | | | | |
| | Timepoint (min.) | | | | | |
| Temperature (° C.) | 15 | 30 | 60 | 120 | 240 | 480 |
| 20 | 95.77 | 91.82 | 87.38 | 81.04 | 70.85 | 56.62 |
| 50 | 79.21 | 74.51 | 65.73 | 42.50 | 6.78 | 2.35 |
| | Compound D (p-Nitro) % Remaining | | | | | |
| | Timepoint (min.) | | | | | |
| Temperature (° C.) | 15 | 30 | 60 | 120 | 240 | 480 |
| 20 | 97.54 | 96.41 | 95.69 | 94.78 | 93.59 | 91.31 |
| 50 | 86.19 | 77.18 | 72.58 | 67.54 | 65.59 | 66.22 |
| | Compound E (NHS) % Remaining | | | | | |
| | Timepoint (min.) | | | | | |
| Temperature (° C.) | 15 | 30 | 60 | 120 | 240 | 480 |
| 20 | 87.24 | 78.45 | 70.03 | 54.46 | 24.99 | 4.50 |
| 50 | 72.20 | 47.31 | 20.75 | 3.50 | 0.10 | 0 |

Note:
% Active Ester Remaining is in comparison to any hydrolysis products observed in the 254 nm spectrum.

### Example 10. Stability of Compounds A-E in DMSO.

### General Experimental Protocol for DMSO Stability Study

To a 4 mL scintillation vial containing Compound A-E (in respective experiments; 4 mg) was added a stir bar followed by 500 μL of anhydrous dimethyl sulfoxide and was allowed to stir at room temperature (22±2° C.) for 4 hours. Timed aliquots at time (T)=15, 30, 60, 120 and 240 minutes were taken, dissolved in water to achieve 2% DMSO composition and analyzed by HPLC-MS using elution method 1 (Table 5).

TABLE 5
Stability of Compounds A-E in DMSO.

| | % Active Ester Remaining | | | | |
|---|---|---|---|---|---|
| | in DMSO at 22 ± 2° C. | | | | |
| | Timepoint (min.) | | | | |
| Active Ester | 15 | 30 | 60 | 120 | 240 |
| Compound A (DCP) | 100 | 99.95 | 99.81 | 99.75 | 99.60 |
| Compound B (DFP) | 99.89 | 99.81 | 99.79 | 99.78 | 99.46 |
| Compound C (TFP) | 96.55 | 94.71 | 93.91 | 92.60 | 88.57 |
| Compound D (p-Nitro) | 99.35 | 98.99 | 99.29 | 99.65 | 99.31 |
| Compound E (NHS) | 97.29 | 97.13 | 96.83 | 94.53 | 91.57 |

Note:
% Active Ester Remaining is in comparison to any hydrolysis products observed in the 254 nm spectrum.

### Example 11. Functional Group Compatibility: Active Ester Reactivity Study with Amino Acids

General Experimental Protocol for Nucleophilic Reactivity Study with Amino Acids

To a 4 mL scintillation vial containing Compound A or C (1 eq.) was added a stir bar followed by 70 μL of HPLC grade acetonitrile. In another 4 mL scintillation vial containing a respective amino acid (1-5 eq.) was added 130 μL HPLC grade water followed by diisopropylethylamine (5 eq.). The solutions were combined and allowed to stir at room temperature (22±2° C.) for 4 hours. Timed aliquots at time (T)=15, 30, 60, 120 and 240 minutes were taken and analyzed by HPLC-MS using elution method 1 (Table 6).

TABLE 6
Functional Group Compatibility: Active Ester Reactivity Study with Amino Acids

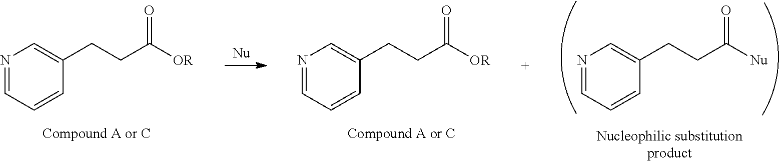

| | Timepoint (min.) | | | | |
|---|---|---|---|---|---|
| Nucleophile (eq.) | 15 | 30 | 60 | 120 | 240 |
| | Compound A (DCP) Reactions: | | | | |
| | % Compound A Remaining (% Nucleophilic Substitution Product) | | | | |
| Z-Ser-OH (1 eq.) | 97.56 | 97.14 | 96.30 | 93.35 | 91.55 |
| | (0) | (0) | (0.08) | (0.23) | (0.44) |
| Z-Arg-OH (1 eq.) | 94.23 | 90.34 | 86.74 | 78.45 | 73.31 |
| | (1.04) | (1.8) | (2.98) | (4.18) | (5.81) |
| N-Acetyl-L-Cysteine | 100 | 99.86 | 99.84 | 99.70 | 82.52 |
| (5 eq.) | (0) | (0.14) | (0.16) | (0.30) | (17.48) |
| | Compound C (TFP) Reactions: | | | | |
| | % Compound C Remaining (% Nucleophilic Substitution Product) | | | | |
| Z-Ser-OH (1 eq.) | 41.75 | 28.90 | 10.96 | 2.89 | 0.12 |
| | (5.90) | (9.02) | (12.30) | (14.11) | (14.81) |
| Z-Arg-OH (1 eq.) | 22.34 | 3.47 | 0.36 | 0 | |
| | (19.78) | (22.24) | (24.43) | (24.57) | |
| N-Acetyl-L-Cysteine | 95.12 | 91.95 | 85.25 | 56.62 | 0 |
| (5 eq.) | (4.88) | (8.05) | (14.75) | (43.38) | (100) |

Note:
% Nucleophilic Substitution Product and % Active Ester Remaining is in comparison to any hydrolysis products observed in the 254 nm spectrum.

### Example 12. Competitive Reactivity Study between L-Lysine and HOPO or HOA Nucleophiles

General Experimental Protocol for Competitive Reactivity Study with L-Lysine and HOPO or HOA Nucleophiles

To a 4 mL scintillation vial containing Compound A (1 eq.) was added a stir bar followed by 70 μL of HPLC grade acetonitrile followed by a respective HOPO or HOA nucleophile (1 eq). In another 4 mL scintillation vial containing L-Lysine monohydrochloride (5 eq.) was added 130 μL HPLC grade water followed by diisopropylethylamine (5 eq.). The solutions were combined and allowed to stir at room temperature (22±2° C.) for 4 hours. Timed aliquots at time (T)=15, 30, 60, 120 and 240 minutes were taken and analyzed by HPLC-MS using elution method 1 (Table 7)

TABLE 7
Competitive Reactivity Study between L-Lysine and HOPO or HOA Nucleophiles.

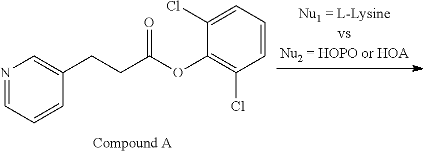 [SMILES: O=C(CCc1cccnc1)Oc1c(Cl)cccc1Cl]

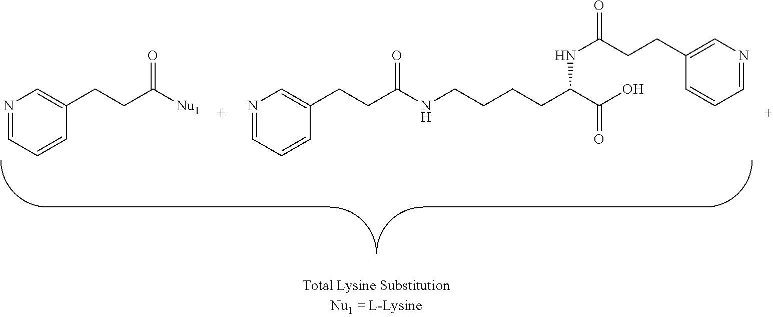

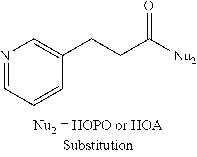

| | Compound A (DCP) Reactions: | | | | |
|---|---|---|---|---|---|
| | % Total Lysine Substitution / % HOPO or HOA Substitution | | | | |
| | (% Compound A Remaining) | | | | |
| Nucleophile | Timepoint (min.) | | | | |
| (eq.) | 15 | 30 | 60 | 120 | 240 |
| L-Lysine (5 eq.) / | 11.67/0 | 25.48/0 | 71.71/0 | 97.99/0 | 100/0 |
| 2- | | | | | |
| Hydroxypyridine- | (88.33) | (74.52) | (28.29) | (2.01) | (0) |
| 1-oxide (1,2- | | | | | |
| HOPO; 1 eq.) | | | | | |
| L-Lysine (5 eq.) / | 34/0.24 | 84.84/0.33 | 97.54/0.83 | 98.99/1.01 | |
| N-Hydroxy-N- | (65.77) | (14.83) | (1.63) | (0) | |
| methyl benzamide | | | | | |
| (1 eq) | | | | | |

Note:
% Total Lysine Substitution, % HOPO or HOA Substitution and % Active Ester Remaining is in comparison to any hydrolysis products observed in the 254 nm spectrum.

### Example 13. Synthesis of Model Compound F Containing A HOPO and Dichlorophenyl Ester

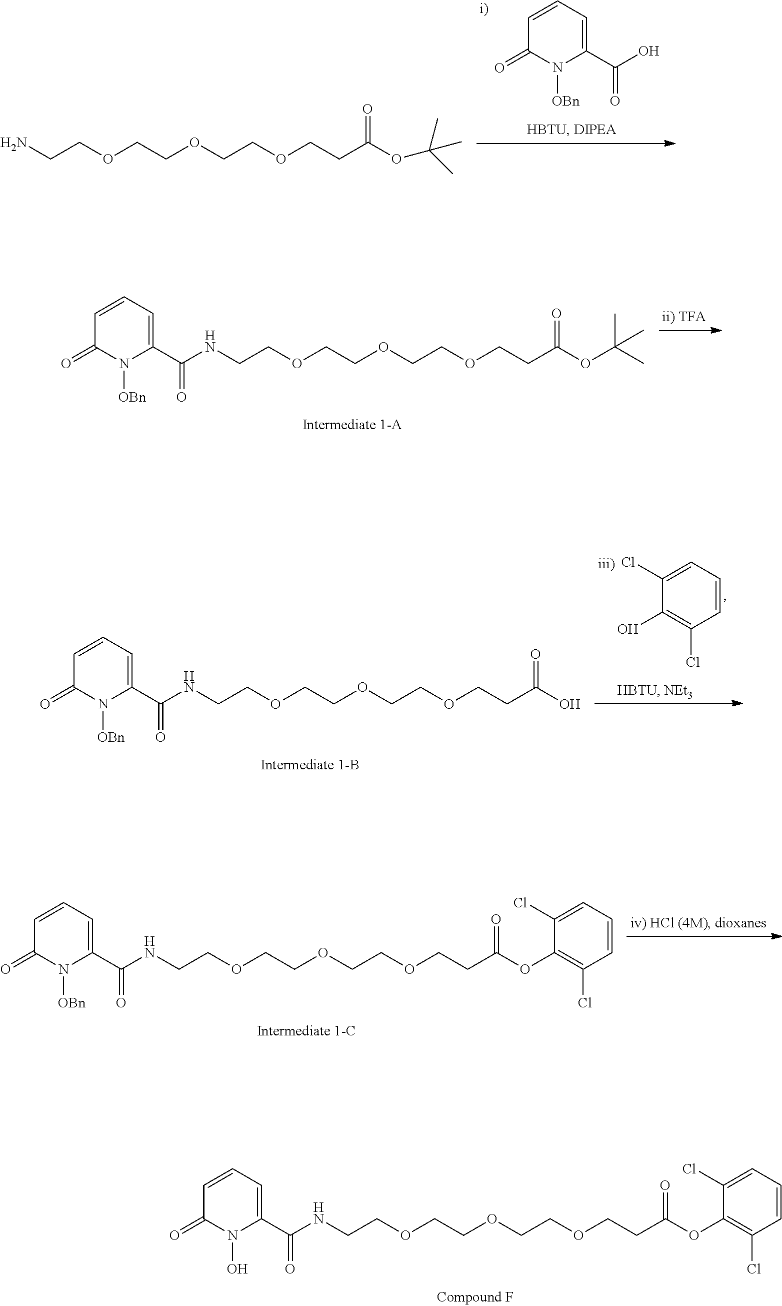

Synthesis of tert-Butyl 3-{2-[2-(2-{[1-(benzyloxy)-6-oxopyridin-2-yl]formamido}ethoxy]ethoxylethoxy}propanoate (Intermediate 1-A)

To a solution of 1-(benzyloxy)-6-oxo-1,6-dihydropyridine-2-carboxylic acid (134 mg, 0.52 mmol) in anhydrous MeCN (2 mL) was added HBTU (203 mg, 0.52 mmol), DIPEA (457 μL, -2.6 mmol) and stirred at RT for 5 min and then the tert-Butyl 12-amino-4,7,10-trioxadodecanoate (150 mg, 0.43 mmol) dissolved in anhydrous MeCN (1 mL). The resulting brown, clear solution was completely soluble and was stirred in a 50° C. oil bath. The reaction was monitored by HPLC-MS and then concentrated under vacuum after 1.5 h. The crude material was then purified on a preparative C18 HPLC column to afford Intermediate 1-A (87 mg, 39%, 97.6% purity) as a pale yellow film. Additionally, 62.9 mg of product in 70% purity was also isolated.

Synthesis of 3-{2-[2-(2-(2-{8 1-(Benzyloxy)-6-oxopyridin-2-yl]formamidolethoxy}ethoxylethoxy}propanoic acid (Intermediate 1-B)

To a solution of Intermediate 1— A (87 mg, 0.17 mmol) in 1 mL of anhydrous DCM was added 2 mL of TFA and stirred at RT. The reaction was monitored by HPLC-MS and then worked up after 2 h 20 min by concentrating to dryness under airstream and then concentrated under vacuum to afford Intermediate 1-B (112 mg, quant) as a clear film as the TFA salt and used in the next step without further purification.

Synthesis of 2,6-Dichlorophenyl 3-{2-2-{[1-(benzyloxy)-6-oxopyridin-2-yl]formamido}ethoxy)ethoxylethoxy}propanoate (Intermediate 1-C)

To a 20 mL scintillation vial containing Intermediate 1-B (48 mg, 0.11 mmol) was added a stir bar, ACN (2.0 mL) followed by NEt₃ (71 μL, 0.52 mmol), HBTU (50 mg, 0.13 mmol) and lastly a solution of 2,6-dichlorophenol (72 mg, 0.43 mmol dissolved in 0.15 mL of ACN). The resulting solution was allowed to stir at RT. The reaction was monitored by HPLC-MS and then concentrated under vacuum after 2 h. The crude material was then purified on a preparative C18 HPLC column to afford Intermediate 1-C (33 mg, 51%) as a clear film.

Synthesis of 2, 6-Dichlorophenyl 3-12-(242-[(1-hydroxy-6-oxopyridin-2-yl)formamidolethoxy]ethoxy)ethoxylpropanoate (Compound F)

To a 1 dram vial containing Intermediate 1-C (5 mg, 8 μtmol) was added a stir bar and then 0.5 mL of HC1 (4 M) in dioxanes. The resulting clear solution was stirred in a 50° C. oil bath.

The reaction was monitored by HPLC-MS and then concentrated under an air stream after 4 h. The crude material was then purified on a preparative C18 HPLC column to afford Compound F (3.3 mg, >90.9% purity, 71%) as a clear film. An aliquot was analyzed by HPLC-MS elution using elution method 2; retention time: 3.60 min; MS (positive ESI): found m/z 502.7 [M+H]⁺; C₂₁H₂₅Cl₂N₂O₈ (calc. 503.10).

### Example 14. Amine Reactivity of Model Compound F

Synthesis of (2S)-2-Amino-6-{3-[2-(2-{2-[(1-hydroxy-6-oxopyridin-2-yl)formamidolethoxy]ethoxy}ethoxy]propanamido}hexanoic acid (Compound G)

To a 1 dram vial containing L-Lysine monohydrate in trace select H₂O (264 μL, 5.0 mg/mL, 10 μmol) and a stir bar was added DIPEA (1.6 μL, 10 μmol) and stirred at RT for ˜1 min. Lastly a solution of Compound F (0.5 mg, 0.9 μmol) in MeCN (50 μL) was added. The resulting solution was stirred at RT and monitored by HPLC-MS after 120 min. An aliquot was analyzed by HPLC-MS elution using elution method 2; retention time of Compound G mass: 0.97 min, 1.03 min (major), 1.60 min; MS (positive ESI): found m/z 486.9, 486.9 and 487.0 [M+H]⁺ for respective retention times; C₂₁H₃₅N₄O₉ (calc. 487.24); unoptimized analytical method and minor amount of a-amine of Lysine to account for the apparent multiple product peaks listed above.

Synthesis of N-Benzyl-3-[2-(2-{2-[(1-hydroxy-6-oxopyridin-2-yl)formamido]ethoxy}ethoxy)ethoxy]propanamide (Compound H)

To a 1 dram vial containing benzyl amine in trace select H₂O (264 μL, 3.7 mg/mL, 9 μmol) and a stir bar was added DIPEA (1.6 μL, 10 μmol) and stirred at RT for ˜1 min. Lastly a solution of Compound F (0.5 mg, 0.9 μmol) in MeCN (50 μL) was added. The resulting solution was stirred at RT and monitored by HPLC-MS after 120 min. An aliquot was analyzed by HPLC-MS elution using elution method 2; retention time of Compound H mass: 2.20 min; MS (positive ESI): found m/z 447.8 [M+H]⁺; C₂₂H₃₀N₃O₇ (calc. 448.21).

TABLE 8
Amine Reactivity of Model Compound F

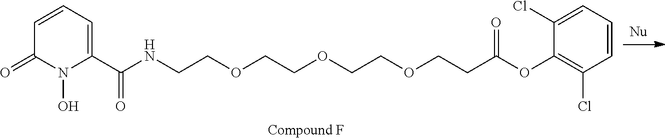 [SMILES: O=C(CCOCCOCCOCCNC(=O)c1cccc(=O)n1O)Oc1c(Cl)cccc1Cl]

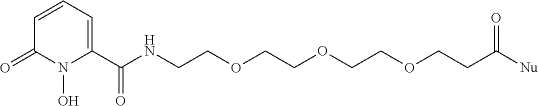

| | % Nucleophilic Substitution Product(s) |
|---|---|
| Nucleophile (10 eq.) | Timepoint (120 min.) |
| L-Lysine | >99% (Compound G) |
| Benzylamine | >99% (Compound H) |

### Example 15. Synthesis of Bis 2,6-Dichlorophenyl Esters

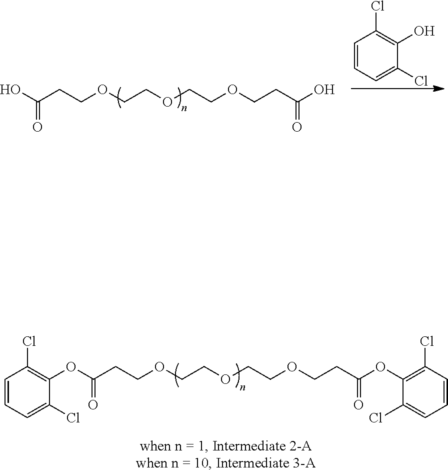

Synthesis of 2, 6-Dichlorophenyl 3-(2-{2-[3-(2,6-dichlorophenoxy)-3-oxopropoxyl]thoxy}ethoxy)propanoate (Intermediate 2-A)

To a 20 mL scintillation vial containing 3-{2-[2-(2-carboxyethoxy)ethoxy]ethoxy}propanoic acid (bis-PEG3-acid, 250 mg, 0.98 mmol) in 3 mL of anhydrous 1,4-dioxanes was added a stir bar and 2,6-dichlorophenol (365 mg, 2.15 mmol). The clear solution was then placed in an ice bath and stirred for 5 minutes. Lastly, N,N′-dicyclohexylcarbodiimide (DCC, 449 mg, 2.15 mmol) was added in 3 mL of anhydrous 1,4-dioxanes in one portion and then the reaction was removed from the ice bath and stirred at room temperature for 6.5 h during which time the reaction progress was monitored by HPLC-MS. Proceeded to add 1 mL of anhydrous DMF which did not fully solubilize the reaction contents and next added HBTU (557 mg, 1.42 mmol) and DIPEA (0.75 mL, 4.31 mmol) and stirred at room temperature for 65 h. The reaction was monitored by HPLC-MS and then worked up by concentration under vacuum to afford a brown oil. The residual DMF remaining was concentrated under an airstream to afford a thick brown oil. The reaction was purified on a preparative C18 HPLC column to afford Intermediate 2 — A (319 mg, 60%) as a pale yellow oil. ¹H NMR (600 MHz, CDCl₃) 7.33 (d, J=8.1 Hz, 4H), 7.11 (t, J=8.1 Hz, 2H), 3.90 (t, J=9.0 Hz, 4H), 3.68-3.62 (m, 8H), 2.95 (t, J=6.0 Hz, 4H).

Synthesis of Bis(2, 6-dichlorophenyl) 4,7,10,13,16,19,22,25,28,31,34,37-dodecaoxatetracontanedioate (Intermediate 3-A)

To a 20 mL scintillation vial containing Bis-PEG12-acid (250 mg, 0.38 mmol) and a stir bar was added a solution of 2,6-dichlorophenol (192 mg, 1.14 mmol in 3 mL of anhydrous 1,4-dioxanes). The clear solution was then stirred at room temperature and DIPEA (397 μL, 2.27 mmol) was added. The solution was then stirred for 5 min and lastly, HBTU (435 mg, 1.11 mmol) was added in one portion and then the mixture was stirred at room temperature for 3.5 h and was found to have went to completion by HPLC-MS. The reaction was worked up by concentration under vacuum to afford a clear residue and purified on a preparative phenyl HPLC column to afford Intermediate 3-A (234 mg, 65%) as a colourless oil. An aliquot was analyzed by HPLC-MS elution using elution method 2; retention time of Intermediate 3-A mass: 5.75 min; MS (positive ESI): found m/z 957.0 [M+Na]⁺; C₄₀H₅₈Cl₄NaO₁₆ (calc. 957.24).

### Example 16. Application to the Synthesis of an Acyclic Hydroxamic Acid Dichlorophenyl Ester Chelate (Compound I and Compound J)

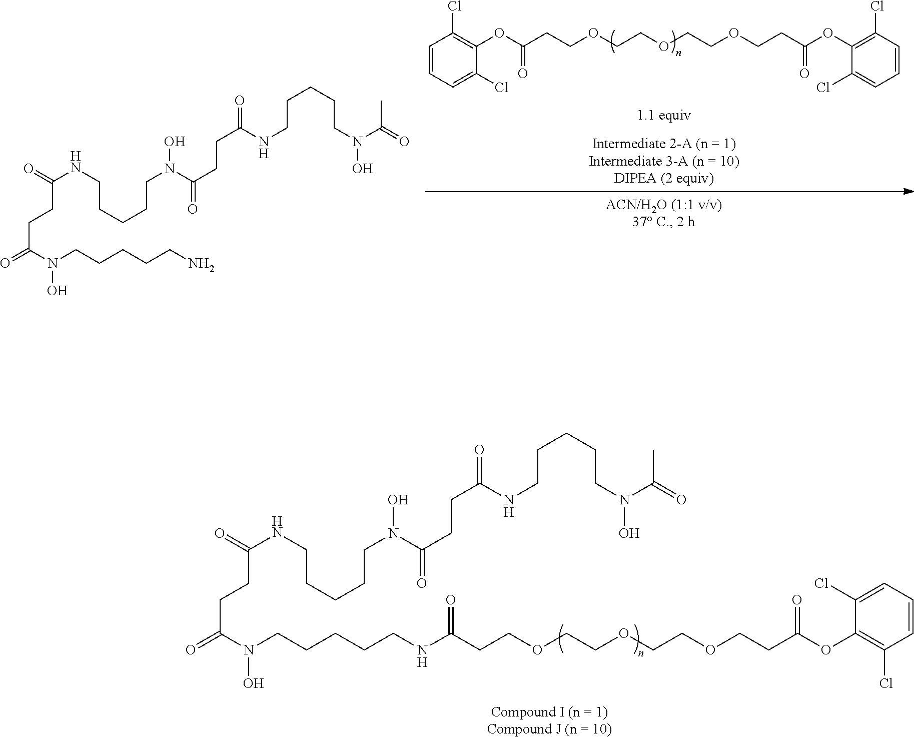

Synthesis of 2,6-Dichlorophenyl 3-{2-[2-(2-{[5-(N-hydroxy-3-{[5-(N-hydroxy-3-{[5-(N-hydroxyacetamido)pentyl]carbamoyl}propanamido)pentyl]carbamoyl}propanamido)pentyl]carbamoyl}ethoxy)ethoxy]ethoxy}propanoate (Compound I)

To a 1 dram vial was loaded DFO mesylate (25 mg, 35 μmol), H₂O (0.631 mL) and a stir bar. Upon dissolution (<5 min) a freshly prepared solution of 2, 6-dichlorophenyl 3-(2-{2-[3-(2,6-dichlorophenoxy)-3-oxopropoxy]ethoxy}ethoxy)propanoate (Intermediate 2-A, 25 mg, 45 μmol dissolved in 0.631 mL of anhydrous MeCN) was added in one portion followed by the addition of DIPEA (14 μL, 82 umol). The reaction vial was capped, teflon taped and stirred in a 37° C. oil bath for 2 h and was monitored by HPLC-MS. The reaction was worked up by concentration under vacuum and then purified on a preparative C18 HPLC column to afford Compound I (5.5 mg, 17%) as a white solid. An aliquot was analyzed by HPLC-MS elution using elution method 4; retention time: 3.60 min; MS (positive ESI): found m/z 937.1 [M+H]⁺; C₄₁H₆₇Cl₂N₆O₁₄ (calc. 937.40).

Synthesis of 2,6-Dichlorophenyl 1-{[5-(N-hydroxy-3-{8 5-(N-hydroxy-3-{[5-(N-hydroxyacetamido)pentyl]carbamoyl}propanamido)pentyl]carbamoyl}propanamido)pentylic carbamoyl}-3,6,9,12,15,18,21,24,27,30,33,36-dodecaoxanonatriacontan-39-oate (Compound J)

To a 1 dram vial was loaded DFO mesylate (25 mg, 35 umol), H 2 O (0.631 mL) and a stir bar. Upon dissolution (<5 min) a freshly prepared solution of Bis(2,6-dichlorophenyl) 15 4,7,10,13,16,19,22,25,28,31,34,37-dodecaoxatetracontanedioate (Intermediate 3-A, 37 mg, 39 μmol dissolved in 0.631 mL of anhydrous MeCN) was added in one portion followed by the addition of DIPEA (12 μL, 70 μmol). The reaction vial was capped, teflon taped and stirred in a 37° C. oil bath for 2 h and was monitored by HPLC-MS. The reaction was worked up by concentration under vacuum and then purified on a preparative C18 HPLC column to afford Compound J (10 mg, 21%) as a white solid. An aliquot was analyzed by HPLC-MS elution using elution method 4; retention time: 3.61 min; MS (positive ESI): found m/z 1333.2 [M+H]⁺; C₅₉H₁₀₃Cl₂N₆O₂₃ (calc. 1333.65).

### Example 17. Stability of Compound J

To an HPLC vial was added a 0.2 mg/mL solution of Compound J dissolved in H₂O (0.1% v/v TFA) at pH=2 and the stability was monitored by HPLC-MS at 20° C. over 16 hours. Timed aliquots at time (T)=0, 30, 60, 120, 180, 240, 480 and 960 minutes were taken and analyzed by HPLC-MS using elution method 4 at 214 nm (Table 9).

TABLE 9
Stability of Compound J at pH = 2

| | % Compound J Remaining | | | | | | | |
|---|---|---|---|---|---|---|---|---|
| | Timepoint (min.) | | | | | | | |
| pH | 0 | 30 | 60 | 120 | 180 | 240 | 480 | 960 |
| 2 | 97.49 | 97.21 | 96.67 | 96.11 | 96.14 | 94.83 | 94.39 | 91.48 |

### Example 18. Reaction of Compound J with L-Lysine

Synthesis of (2S)-2-Amino-6-(1{[5-(N-hydroxy-3-{[5-(N-hydroxy-3-{[5-(N-hydroxyacetamido)pentyl]carbamoyl}propanamido)pentyl]carbamoyl}propanamido)pentyl]c arbamoyl}-3,6,9,12,15,18,21,24,27,30,33,36-dodecaoxanonatriacontan-39-amido)hexanoic acid (Compound K)

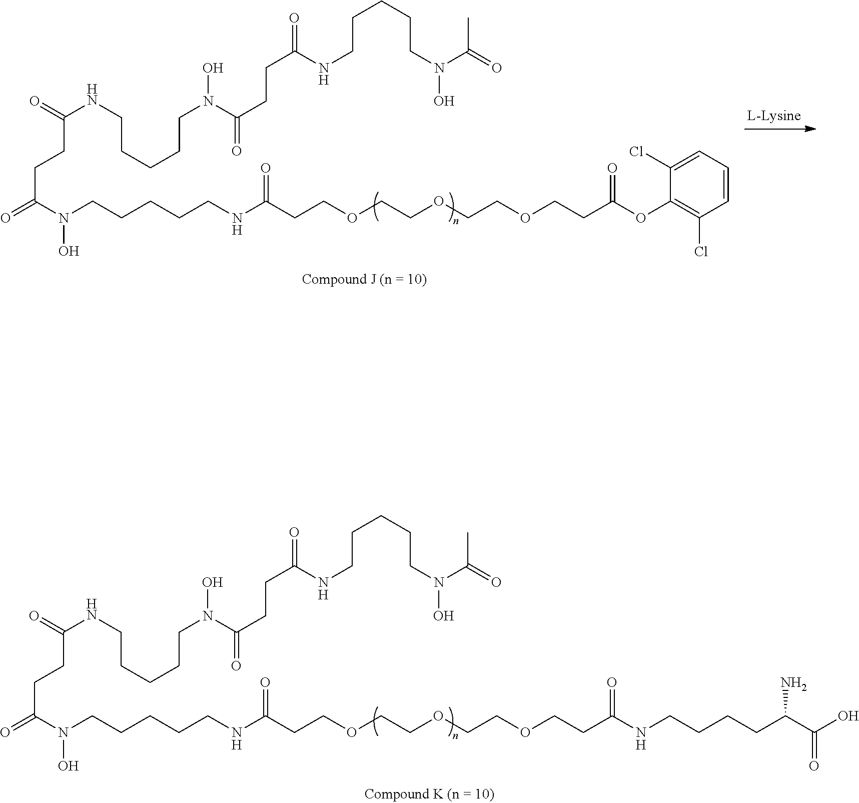

To a 4 mL scintillation vial containing Compound J (0.60 mg, 0.44 i.tmol, 1 eq.) was added a stir bar. In another 4 mL scintillation vial containing L-Lysine monohydrochloride (0.41 mg, 2.2 μmol, 5 eq.) was added 100 μL of a pH 9 SABST buffer solution (SABST=a sodium acetate (0.1 M) buffered saline solution with 0.01% Tween 80, brought to pH 9 with 0.1 M solution of Na₂CO₃). The solutions were combined, and the pH was confirmed to be pH and the resulting solution was allowed to stir at room temperature (22±2° C.) for 4 hours. An aliquot was analyzed by HPLC-MS elution using elution method 2; formation of Compound K was observed as the major product at retention time: 2.19 min; MS (positive ESI): found m/z 1317.4 [M+H]⁺; C₅₉H₁₁₃Na₈O₂₄ (calc. 1317.79).

### Example 19. Synthesis of Macrocyclic HOPO Dichlorophenyl Ester Chelates

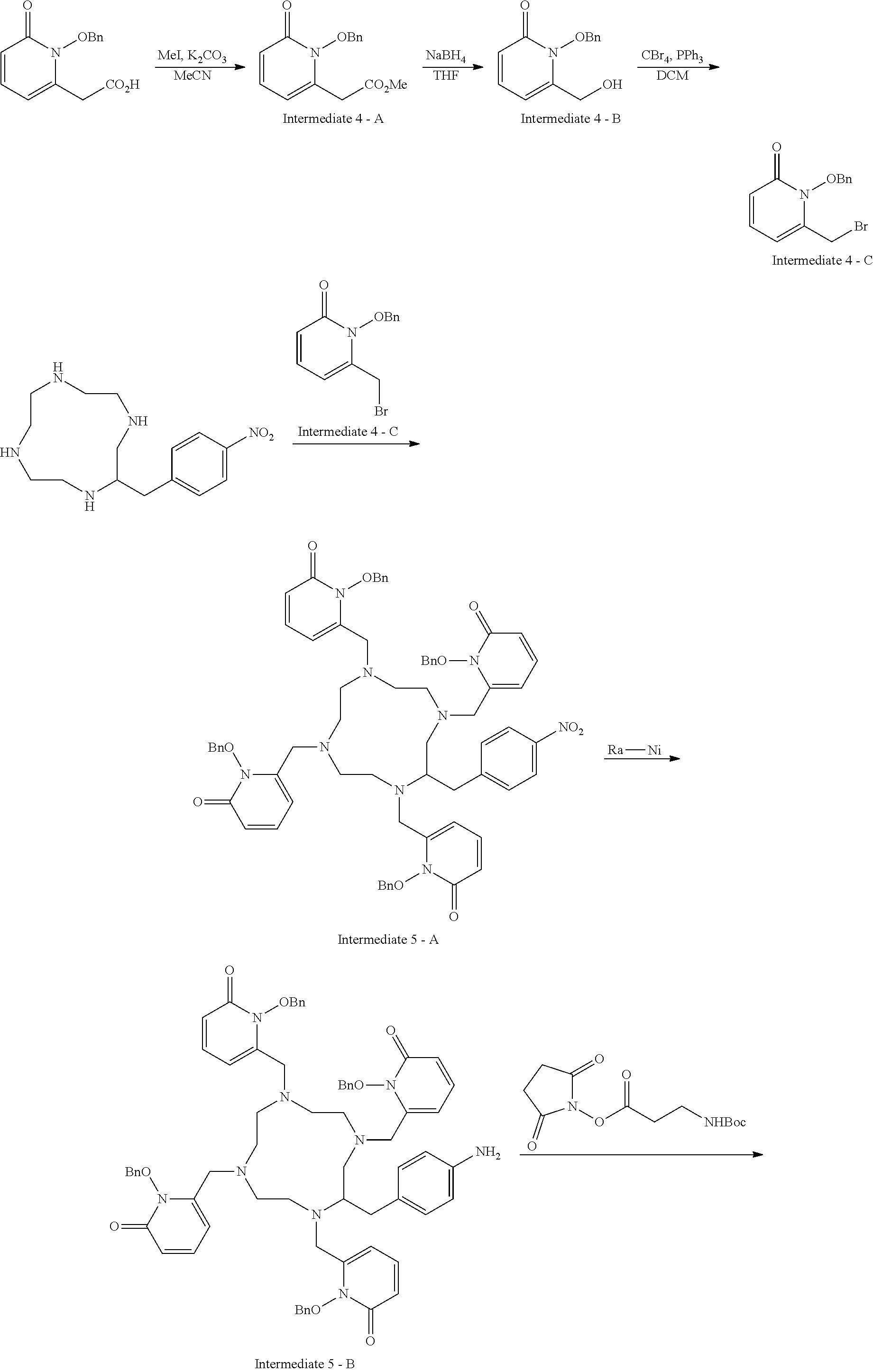

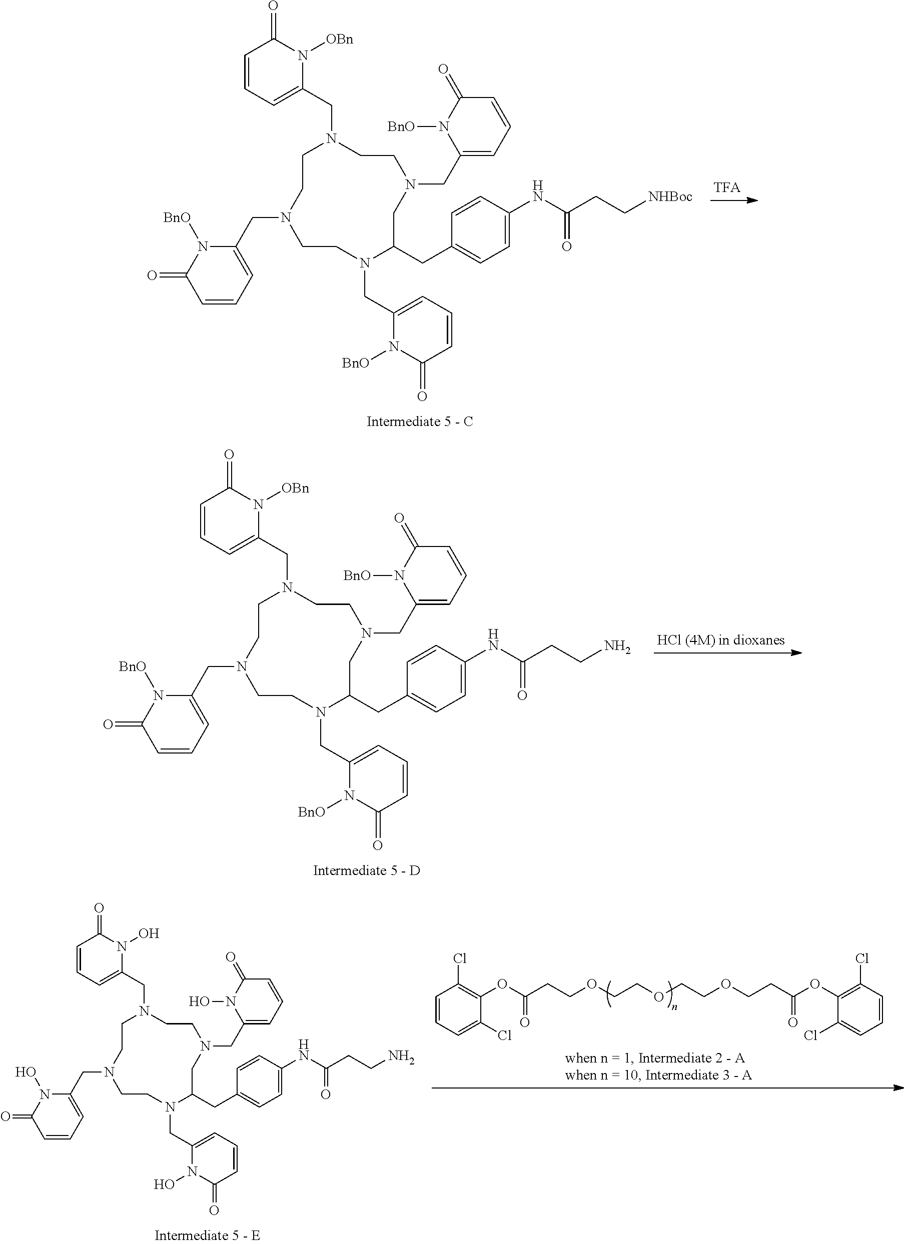

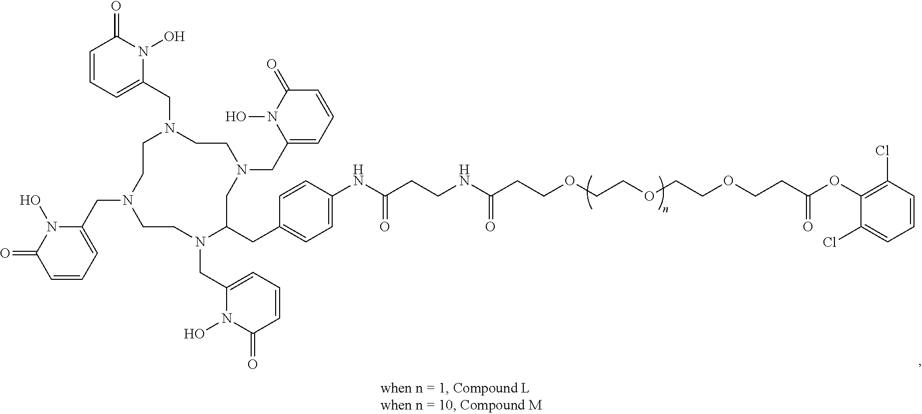

Step 1: Synthesis of Methyl 1-(benzyloxy)-6-oxo-1,6-dihydropyridine-2-carboxylate (Intermediate 4-A)

A 20 mL scintillation vial was charged with 1-(benzyloxy)-6-oxo-1,6-dihydropyridine-2-carboxylic acid (200 mg, 815 μmol) followed by potassium carbonate (225 mg, 1.63 mmol) and 5 mL anhydrous acetonitrile and 5 mL anhydrous tetrahydrofuran. Iodomethane (110 μL, 1.77 mmol) was added and the vial was sealed and stirred at 40° C. for 16 h. An additional portion of iodomethane (55 μL 885 μmol) was then added and the reaction was continued for an additional 24 h. The solids were then removed by filtration and the filtrate was concentrated to dryness under reduced pressure. The residue was dissolved in 4 mL dichloromethane and residual solids were removed by a 2^{nd} filtration. The mother liquor was co-evaporated with 2×3 mL acetonitrile to afford methyl 1-(benzyloxy)-6-oxo-1,6-dihydropyridine-2-carboxylate (Intermediate 4-A) as a clear yellow oil (214 mg, 98% purity by HPLC, 99% yield).

Step 2: Synthesis of 1-(Benzyloxy)-6-(hydroxymethyl)-1,2-dihydropyridin-2-one (Intermediate 4-B)

A 25 mL round bottom flask was charged with 1-(benzyloxy)-6-oxo-1,6-dihydropyridine-2-carboxylic acid methyl ester (Intermediate 4-A, 214 mg, 829 μmol) followed by NaBH₄ (385 mg, 9.95 mmol) and 8 mL anhydrous tetrahydrofuran. The flask was then affixed with a reflux condenser and a nitrogen balloon and heated to reflux for 16 h. The reaction mass was then cooled to 0-5° C. and quenched with the slow addition of 5 mL of methanol. The mixture was concentrated to dryness under reduced pressure and then dissolved in a mixture of dichloromethane and water. 2 mL of saturated ammonium chloride solution was added, and the phases were separated by separatory funnel. The aqueous phase was extracted with 4×20 mL dichloromethane, the organics were combined and dried over Na₂SO₄ (s). Solids were removed by filtration, washed with 3×20 mL dichloromethane and the filtrate was concentrated under reduced pressure to afford 1-(benzyloxy)-6-(hydroxymethyl)-1,2-dihydropyridin-2-one (Intermediate 4-B) as a waxy white solid (144 mg, 85% purity by HPLC, 64% yield).

### Step 3: Synthesis of 1-(Benzyloxy)-6-(bromomethyl)pyridine-2-one (Intermediate 4-C)

A 20 mL scintillation vial was charged with 1-(benzyloxy)-6-(hydroxymethyl)-1,2-dihydropyridin-2-one (Intermediate 4-B, 63 mg, 272 μmol) followed by tetrabromomethane (135 mg, 409 μmol) and 2 mL of anhydrous dichloromethane. The mixture was then cooled in an ice-water bath. After 10 minutes of cooling, triphenylphosphine (110 mg, 409 μmol) was added portion wise as a solid over 10 mins. After another 10 minutes the reaction was checked by TLC and confirmed to be complete. The reaction was quenched with 0.5 mL saturated sodium sulfite (Na₂SO₃) solution and allowed to stir at room temperature for 30 mins. The reaction was then transferred to a separatory funnel, extracted into dichloromethane and the organics were dried over Na₂SO₄ (s). Solids were removed by filtration and the mother liquor was concentrated under reduced pressure to a residue. Purification by flash column chromatography on silica (eluent: 30% toluene in ethyl acetate) afforded 1-(benzyloxy)-6-(bromomethyl)pyridine-2-one (Intermediate 4-C) as a clear viscous oil that solidified to a white film on standing (63 mg, 75%).

Step 2: Synthesis of 1-(Benzyloxy)-6-{[4,7,10-tris({[1-(benzyloxy)-6-oxopyridin-2-yl]methyl})-6-[(4-nitrophenyl)methyl]-1,4,7,10-tetraazacyclododecan-1-yl]methyl}pyridin-2-one (Intermediate 5-A)

To a 20 mL scintillation vial containing Intermediate 4-C (112 mg, 0.382 mmol), 2-[(4-nitrophenyl)methyl]-1,4,7,10-tetraazacyclododecane (25 mg, 0.076 mmol) and a stir bar was added K₂CO₃ (63 mg, 0.459 mmol) and anhydrous MeCN (3 mL). The resulting solution was stirred in a 75° C. oil bath for 65 h. The reaction was monitored by HPLC-MS and worked up by filtration through a fritted filter. The filtered solids were washed with MeCN and then the filtrate was concentrated under vacuum and purified on a preparative C18 HPLC column to afford Intermediate 5-A (120 mg, quant.) as a pale yellow film as the TFA salt.

Step 3: Synthesis of 6-({6-[(4-Aminophenyl)methyl]-4,7,10-tris({[1-(benzyloxy)-6-oxopyridin-2-yl]methyl})-1,4,7,10- tetraazacyclododecan-1-yl}methyl)-1-(benzyloxy)pyridin-2-one (Intermediate 5-B)

A well shaken Ra-Ni 2800 slurry in water (150 μL) was transferred to a 20 mL scintillation vial containing 4 mL of HPLC grade water. The mixture was swirled, allowed to settle and then the water was decanted out (leaving a thin layer on top) and then an additional 4 mL water was used to repeat this wash process. Upon decanting, a 2×4 mL MeOH wash then decant sequence was performed. Lastly, 1 mL of 1:1 THF/MeOH was added, along with a stir bar. Then Intermediate 5-A (20 mg, 0.014 mmol) was added as a solution in 0.5 mL (THF/MeOH, 1:1) and the suspension was then cycled 3× (vacuum for ˜30 seconds then H₂ atmosphere/balloon pressure for ˜30 seconds) and the balloon was left on the reaction and it was left to stir at room temperature for 2.5 h. The reaction was monitored by HPLC-MS and worked up by filtering through a 0.2 μm syringe filter. The reaction vial was washed with an additional 2 mL MeOH and filtered through the syringe filter as well. The combined filtrate was then concentrated under vacuum to afford Intermediate 5-B (19.4 mg, 94%) as a pale yellow film.

Step 4: Synthesis of tert-Butyl N-{2-[(4-{[1,4,7,10-tetrakis({[1-(benzyloxy)-6-oxopyridin-2-yl]methyl})-1,4,7,10-tetraazacyclododecan-2-yl]methyl}phenyl)carbamoyl]ethyl}carbamate (Intermediate 5-C)

To a 20 mL scintillation vial containing Intermediate 5-B (131 mg, 0.077 mmol) was added anhydrous DMF (5 mL) and a stir bar. Next DIPEA (161 μL, 0.93 mmol) was added in one portion followed by DMAP (9.5 mg, 0.077 mmol). The vessel purged with N₂ and then the reaction was stirred at room temperature for 5 min. A freshly dissolved solution of Boc-beta-Ala-OSu (135 mg, 0.463 mmol) in anhydrous DMF (0.5 mL) was added under N₂ atmosphere and then the reaction was stirred in a 50° C. oil bath. After 45 min the reaction progress was monitored by HPLC-MS and primarily starting material along with ˜10% product formation was observed so DMAP (20 mg, 0.164 mmol) and additional Boc-beta-Ala-OSu (135 mg, 0.463 mmol) were added. The reaction was stirred at 50° C. for an additional 18 h. The reaction was worked up by concentration under vacuum and purified on a preparative C18 HPLC column to afford Intermediate 5-C (45 mg, 29%, 76% purity) as a clear film as the TFA salt.

Step 5: Synthesis of 3-Amino-N-(4-{[1,4,7,10-tetrakis({[1-(benzyloxy)-6-oxopyridin-2-yl]methyl})-1,4,7,10-tetraazacyclododecan-2-yl]methyl}phenyl)propenamide (Intermediate 5-D)

To a 20 mL vial containing Intermediate 5-C (14.5 mg, 0.0090 mmol) was added a stir bar and anhydrous DCM (1 mL) and cooled in an ice bath and then trifluoroacetic acid (2 mL) was added and the reaction was stirred for 30 min at room temperature and the reaction progress was monitored by HPLC-MS. The reaction was worked up by concentration under a nitrogen stream in a fume hood and then further dried under vacuum to afford Intermediate 5-D (22 mg, quant) as a clear film as the TFA salt. This material was used in the subsequent step without further purification.

Step 6: Synthesis of 3-Amino-N-[4-({1,4,7,10-tetrakis[(1-hydroxy-6-oxopyridin-2-yl-methyl]-1,4,7,10-tetraazacyclododecan-2-yl}methyl)phenyl]propenamide (Intermediate 5-E)

To a 20 mL scintillation vial containing Intermediate 5-D (10 mg, 0.0067 mmol) was added a stir bar and 2 mL of HC1 (4 M) in dioxanes. The reaction was stirred in a 50° C. oil bath for 1.5 h and the reaction progress was monitored by HPLC-MS. The reaction was then worked up by concentration under a nitrogen stream and then further dried under vacuum to afford Intermediate 5-E (10 mg, quant) as a pale yellow solid. This material was used in the subsequent step without further purification.

Step 7: Synthesis of 2,6-Dichlorophenyl 3-[2-(2-{2-[(2-{[4-({1,4,7,10-tetrakis[(1-hydroxy-6-oxopyridin-2-yl)methyl]-1,4,7,10-tetraazacyclododecan-2-yl}methyl)phenyl]carbamoyl}ethyl)carbamoyl]ethoxy}ethoxy)ethoxy]propanoate (Compound L)

To a 20 mL vial containing Intermediate 5-E in ACN/H₂₀ Trace Select grade (1:1 v/v, 800 μL, ˜8 mg, 0.0053 mmol) was added a stir bar followed by DIPEA (46 μL, 0.26 mmol) and then lastly a solution of Intermediate 2-A (15 mg, 0.027 mmol) in MeCN (400 μL). The reaction was stirred for 1 h at room temperature and then monitored by HPLC-MS. The reaction was worked up by cooling in an ice bath and then adding 50 μL of TFA over ˜30 seconds followed by concentration under vacuum to dryness. The crude was then purified on a preparative C18 HPLC column to afford Compound L (0.7 mg, 7%, ≥81% purity) as a white solid as the TFA salt following lyophilization. An aliquot was analyzed by HPLC-MS elution using elution method 2; retention time: 3.07 min; MS (positive ESI): found m/z 1217.37 [M+H]⁺; C₅₈H₇₁Cl₂N₁₀O₁₅ (calc. 1217.45).

Step 8: Synthesis of 2,6-Dichlorophenyl 1-[(2-{[4-({1,4,7,10-tetrakis[(1-hydroxy-6-oxopyridin-2-yl(methyl]-1,4,7,10-tetraazacyclododecan-2-yl}methyl)phenyl]carbamoyl}ethyl)carbamoyl]-3,6,9,12,15,18,21,24,27,30,33,36-dodecaoxanonatriacontan-39-oate (Compound M)

To a 20 mL scintillation vial containing Intermediate 5-E (˜9.0 mg, 0.0080 mmol) in ACN/H₂O Trace Select grade (1:1 v/v, 900 μL/˜1 mg) was added a stir bar followed by DIPEA (70 μL, 0.040 mmol) and then lastly a solution of Intermediate 3-A (37 mg, 0.040 mmol) in MeCN (374 μL). The reaction was stirred for 40 min at room temperature and then monitored by HPLC-MS. The reaction was worked up by cooling in an ice bath and then adding 90 μL of TFA followed by concentration under vacuum to dryness. The crude was then purified on a preparative C18 HPLC column to afford Compound M (1.2 mg, 6%, ≥68% purity) as a white solid as the TFA salt following lyophilization. An aliquot was analyzed by HPLC-MS elution using elution method 2; retention time: 3.43 min; MS (positive ESI): found m/z 1635.79 [M+Na]⁺; C₇₆H₁₀₆Cl₂N₁₀NaO₂₄ (calc. 1635.67).

### Example 20. Synthesis of (2R)-5-(2,6-Di chl orophenoxy)-5-oxo-2-[4,7,10-tris(carboxymethyl)-1,4,7,10-tetraazacyclododecan-1-yl]pentanoic acid (Compound N)

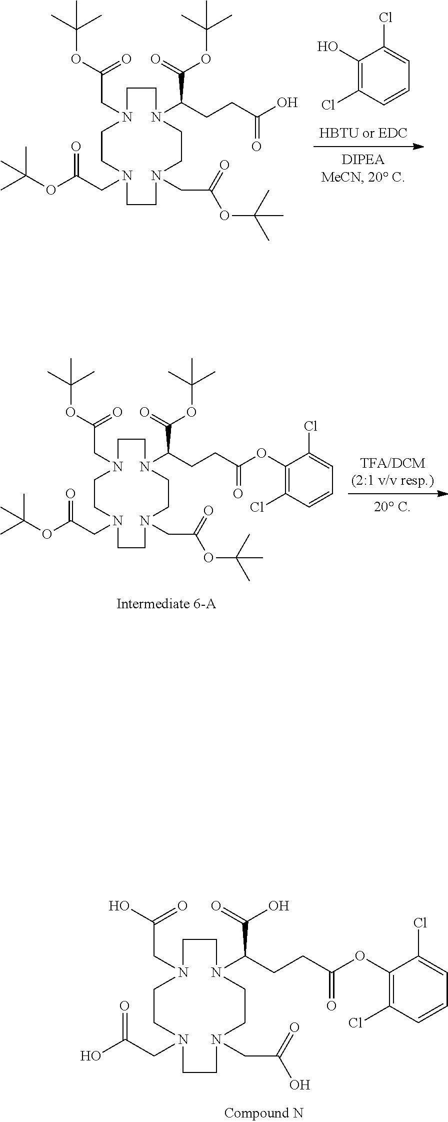

Step 1: 1-tert-Butyl 2,6-dichlorophenyl (2R)-2-{4,7,10-tris[2-(tert-butoxy)-2-oxoethyl]-1,4,7, 10-tetraazacyclododecan-1-yl} pentanedioate (Intermediate 6-A)

To a 20 mL vial containing DOTAGA(tBu)₄ (50 mg, 71.3 μmol), stir bar, anhydrous MeCN (0.5 mL) was added HBTU (42 mg, 110 μmol) and then the reaction was stirred for 5 min at RT. Next a solution of 2,6-dichlorophenol (59 mg, 364 μmol) in 0.50 mL of anhydrous MeCN with DIPEA (62 μL, 364 μmol) was added and then the resulting pale yellow solution was stirred at RT.

The reaction was monitored by HPLC-MS and then concentrated under vacuum after 1.5 h. The crude material was then purified on a preparative C18 HPLC column to afford Intermediate 6-A (23 mg, 30%) as a clear film as the TFA salt.

Alternatively, Intermediate 6-A was also prepared in analogy to above using EDC.HC1 as the coupling agent.

Step 2: Synthesis of (2R)-5-(2,6-Dichlorophenoxy)-5-oxo-2-[4,7,10-tris(carboxymethyl)-1,4,7,10-tetraazacyclododecan-1-yl]pentanoic Acid (Compound N)

To a 20 mL vial containing Intermediate 6-A (19 mg, 17.7 μmol) and a stir bar was added 1 mL anhydrous DCM and 2 mL of TFA. The resulting solution was capped and stirred at 20° C. and the reaction progress was monitored by HPLC-MS. After 43 h the reaction was then worked up by concentration to dryness under a stream of air. The crude was then dissolved in H 2 O (3 mL), frozen and lyophilized to afford Compound N (22.5 mg, 62%, 92.9% purity) as a white fluffy solid as the TFA salt following lyophilization. An aliquot was analyzed by HPLC-MS elution using elution method 2 having dissolved in 0.1% TFA/H₂O; retention time: 2.24 min; MS (positive ESI): found m/z 620.9 [M+H]⁺; C₂₅H₃₅Cl₂N₄O₁₀ (calc. 621.18).

### Example 21. Synthesis of Antibody Conjugates Compound O and Compound P

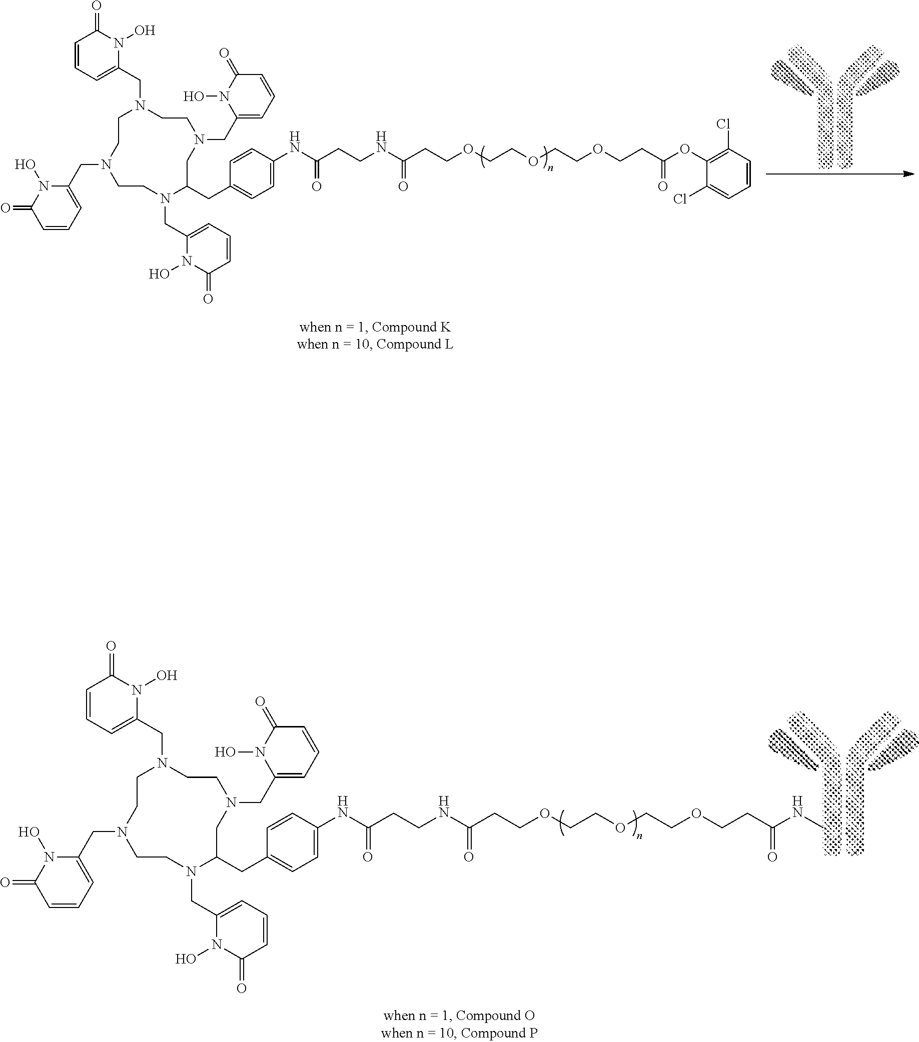

A 1.5 mL Eppendorf was loaded with an antibody (humanized mAb anti-IGF-1R; 9.7 nmol, 1.1 mL in SABST=a sodium acetate (0.1 M) buffered saline solution with 0.01% Tween 80) and sodium bicarbonate buffer (110 μL, 0.1 M). Compound L was added (58.2 μL, 58.2 nmol at a c=1 nmol/μL in 0.001 M HCl). The reaction was incubated at room temperature for 100 min. The reaction was then purified to remove unreacted chelate by G50 column using SAB ST as eluent to afford Compound 0 which was sampled by SEC-HPLC elution method 1 and Nano-drop (˜71% yield). A CAR of 0.80 was determined by MALDI-MS. In analogy to the above, a 6-fold excess of Compound M was reacted with humanized mAb anti-IFG-1R for 120 min at room temperature to afford Compound P which was sampled by SEC-HPLC elution method 1 and Nano-drop (˜78% yield). A CAR of 0.92 was determined by MALDI-MS.

### Example 22. Synthesis of Model Compound Q Containing a 3,2-HOPO and 2,6-Dichlorophenyl Ester

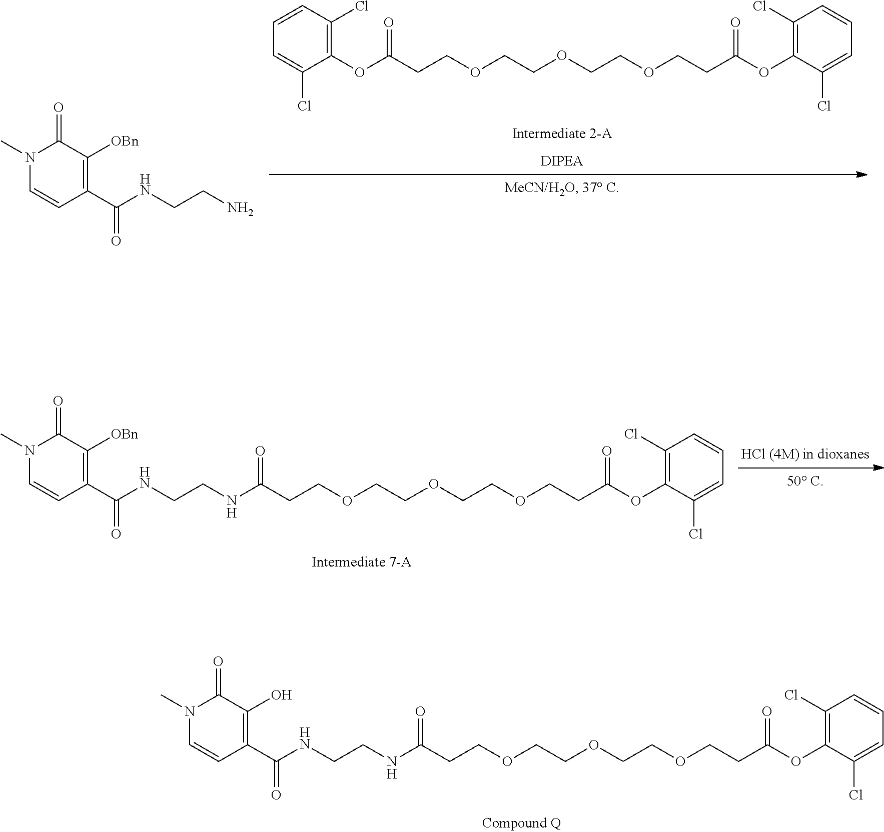 [SMILES: O=C(CCOCCOCCOCCC(=O)Oc1c(Cl)cccc1Cl)Oc1c(Cl)cccc1Cl ; Cn1ccc(C(=O)NCCN)c(OCc2ccccc2)c1=O ; Cn1ccc(C(=O)NCCNC(=O)CCOCCOCCOCCC(=O)Oc2c(Cl)cccc2Cl)c(OCc2ccccc2)c1=O ; Cn1ccc(C(=O)NCCNC(=O)CCOCCOCCOCCC(=O)Oc2c(Cl)cccc2Cl)c(O)c1=O]

Synthesis of 2,6-Dichlorophenyl 3-[2-(2-{(2-{[3-(benzyloxy)-1-methyl-2-oxopyridin-4-yl)formamidolethyl}carbamoyl]ethoxy}ethoxy)ethoxy]propanoate (Intermediate 7-A)

To a solution of N-(2-aminoethyl)-3-(benzyloxy)-1-methyl-2-oxo-1,2-dihydropyridine-4-carboxamide (5.4 mg, 12.9 μmol) in MeCN (250 μL) and water (250 μL) was added a solution of Intermediate 2-A (7.7 mg, 14.2 μmol) in MeCN (250 μL). Lastly DIPEA (4.5 μL, 25.7 μmol) was added and the resulting solution was stirred in a 37° C. oil bath. The reaction was monitored by HPLC-MS and additional DIPEA (4.5 μL, 25.7 μmol) was added after 1 h. The reaction was worked up by concentration under vacuum after 1.5 h.

The crude material was then purified on a preparative C18 HPLC column to afford Intermediate 7-A (4 mg, 44%, 95% purity) as a clear film.

Synthesis of 2,6-Dichlorophenyl 3-[2-(2-{2-([2-{[(3-hydroxy-1-methyl-2-oxopyridin-4-yl]formamido}ethyl)carbamoyl]ethoxy}ethoxy)ethoxy]propanoate (Compound Q)

To a 20 mL scintillation vial containing Intermediate 7-A was added 1 mL HCl (4 M) in dioxanes. The resulting solution was stirred in a 50° C. oil bath for 1.5 h and the reaction was monitored by HPLC-MS. The reaction was then worked up by concentration under an air stream. The crude product was then purified on a preparative C18 HPLC column to afford Compound Q (1.8 mg, 54%, 99% purity) as a clear film. An aliquot was analyzed by HPLC-MS using elution method 2; retention time: 3.38 min; MS (positive ESI): found m/z 587.7 [M+H]⁺; C₂₅H₃₂Cl₂N₃O₉(calc. 588.15).

### Example 23. Reaction of 2,6-Dichlorophenyl Ester with an O-Benzyl Hydroxylamine

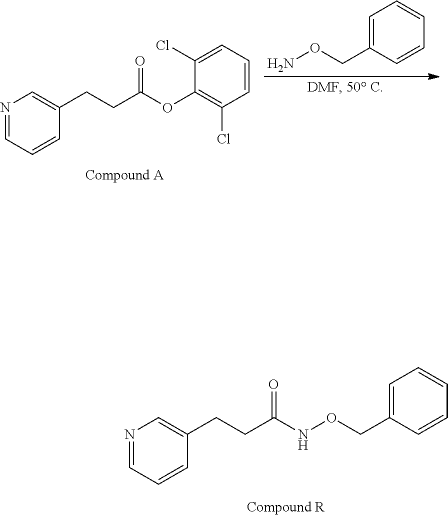

To a 4 mL scintillation vial containing Compound A (5.0 mg, 0.02 mmol, 1 eq.) was added a stir bar followed by O-benzylhydroxylamine hydrochloride (14 mg, 0.08 mmol, 5 eq.) and anhydrous DMF (200 μL. Lastly DIPEA (14.86 μL, 0.08 mmol, 5 eq.) was added and the solution was stirred in a 50° C. oil bath. The reaction was monitored by HPLC-MS using elution method 2 and was found to have went to >98% conversion forming Compound R; retention time: 1.59 min; MS (positive ESI): found m/z 256.9 [M+H]⁺; C₁₅H₁₇N₂O₂ (calc. 257.13).

### Example 24. Conjugation of 2,6-Dichlorophenyl Esters to an Amine Functionalized Stationary Phase

To a round bottomed flask is charged 3-aminopropyl bonded silica gel, 2,6-Dichlorophenyl Ester (Compound L or Compound I) in a ratio of 1:3 to 1:10, DIPEA (10 equiv) and DMF (in analogy to alternative immobilizations described in ppl. Radiat. Isot. 2020, 164, 109264 and J. Sep. Sci. 2015, 38, 60). The resulting mixture is stirred under argon at a temperature ranging from 37-100° C. for 2 to 16 h. The resulting HOPO or HOA functionalized silica gel is filtered and washed successively with DMF, acetone, methanol, water and HCl (0.1 M). Lastly the HOPO or HOA functionalized silica gel is dried under vacuum and then the extent of immobilization is quantified by elemental analysis. The HOPO or HOA functionalized silica is subsequently packed in cartridges and columns of varying size.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

What is claimed is:

1. A method of preparing a conjugate of formula I:
A-L-C(═O)—B (I),
wherein
A is H, heteroalkyl, 5-20 membered heterocycloalkyl, aryl, or heteroaryl, each of heteroalkyl, 5-20 membered heterocycloalkyl, aryl, and heteroaryl being optionally substituted;
L is a linker selected from the group consisting of C₁₋₅₀ alkyl, C₁₋₅₀ heteroalkyl, C₃₋₅₀ cycloalkyl, C₄₋₂₀ cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted; and
B is a moiety comprising an amine unit,
wherein the method comprises:
providing a compound of A-L-C(═O)-M-X, in which M is O or S and X is 2,6-dichlorophenyl; and
conjugating the compound of A-L-C(═O)-M-X with a molecule comprising an amine unit to afford the conjugate of formula I.

2. The method of claim 1, wherein A is a heteroalkyl group comprising one or more hydroxamic acid units, a heteroalkyl group substituted directly or indirectly with one or more hydroxypyridinone units, or a 5-20 membered heterocycloalkyl group substituted directly or indirectly with one or more acetic acid or hydroxypyridinone units.

3. The method of claim 2, wherein A is a heteroalkyl group comprising one or more hydroxamic acid units, wherein the heteroalkyl group is optionally substituted with one or more oxo (═O).

4. The method of claim 3, wherein A has the structure shown below:

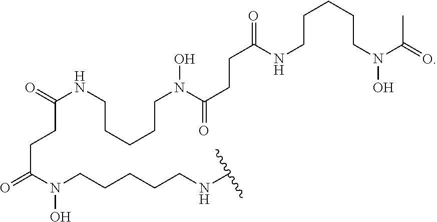

5. The method of claim 2, wherein A is a heteroalkyl group substituted directly or indirectly with one or more hydroxypyridinone units, wherein the heteroalkyl group comprises two or more nitrogen atoms.

6. The method of claim 5, wherein A has the structure shown below:

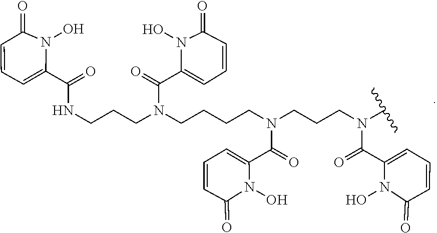 [SMILES: *N(CCCN(CCCCN(CCCNC(=O)c1cccc(=O)n1O)C(=O)c1cccc(=O)n1O)C(=O)c1cccc(=O)n1O)C(=O)c1cccc(=O)n1O]

7. The method of claim 2, wherein A is a 5-20 membered heterocycloalkyl group substituted directly or indirectly with one or more hydroxypyridinone units, wherein the 5-20 membered heterocycloalkyl group comprises three or more nitrogen atoms.

8. The method of claim 7, wherein A has the structure shown below:

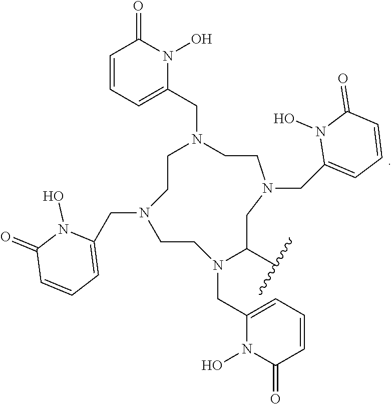 [SMILES: *C1CN(Cc2cccc(=O)n2O)CCN(Cc2cccc(=O)n2O)CCN(Cc2cccc(=O)n2O)CCN1Cc1cccc(=O)n1O]

9. The method of any one of claims 1-8, wherein L is optionally substituted Ci-so alkyl, optionally substituted C₁₋₅₀ heteroalkyl, C═O, —NH—(C═O)—, or a combination thereof, wherein the optionally substituted C₁₋₅₀ heteroalkyl comprises one or more oxygen atoms.

10. The method of claim 9, wherein L comprises 3-20 polyethylene glycol (PEG) units.

11. The method of any one of claims 1-10, wherein B is a moiety formed from lysine, an amine functionalized stationary phase, a peptide, an antibody, or an antigen-binding fragment of an antibody.

12. The method of any one of claims 1-11, wherein B is an antibody, or an antigen-binding fragment thereof, that has a lysine unit conjugated with a compound of A-L-C(═O)—O—X to form the conjugate of A-L-C(═O)—B.

13. The method of claim 1, wherein A is a heteroalkyl group comprising one or more hydroxamic acid units, a heteroalkyl group substituted directly or indirectly with one or more hydroxypyridinone units, or a 5-20 membered heterocycloalkyl group substituted directly or indirectly with one or more acetic acid or hydroxypyridinone units;
wherein L is optionally substituted C₁₋₅₀ alkyl, optionally substituted C₁₋₅₀ heteroalkyl, C═O, —NH—(C═O)—, or a combination thereof, wherein the optionally substituted Ci-so heteroalkyl comprises one or more oxygen atoms; and
B is an antibody, or an antigen-binding fragment thereof, that has a lysine unit conjugated with a compound of A-L-C(═O)—O—X to form the conjugate of A-L-C(=O)—B.

14. The method of claim 13, wherein A is a 5-20 membered heterocycloalkyl group substituted directly or indirectly with one or more hydroxypyridinone units, wherein the 5-20 membered heterocycloalkyl group comprises three or more nitrogen atoms;
wherein L is optionally substituted C₁₋₅₀ heteroalkyl, comprising one or more oxygen atoms; and
B is an antibody, or an antigen-binding fragment thereof, that has a lysine unit conjugated with a compound of A-L-C(═O)—O—X to form the conjugate of A-L-C(═O)—B.

15. The method of claim 1, wherein the compound of A-L-C(═O)-M-X is one of the following: 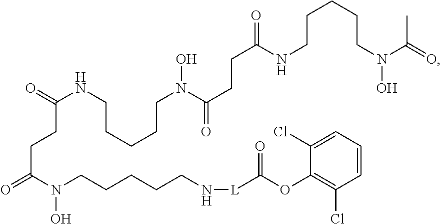
in which L is a linker selected from the group consisting of C₁₋₅₀ alkyl, C₁₋₅₀ heteroalkyl, C₃₋₂₀ cycloalkyl, C₄₋₅₀ cycloalkenyl, C₃₋₂₀ heterocycloalkyl, aryl, heteroaryl, C═O, —NR-(C═P)—, and a combination thereof, R being H or C₁₋₆ alkyl, and each of alkyl, heteroalkyl, cycloalkyl, cycloalkenyl, heterocycloalkyl, aryl, and heteroaryl being optionally substituted; 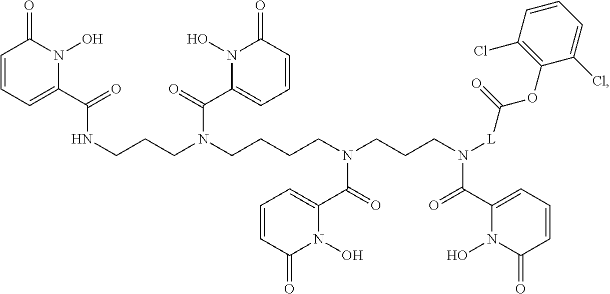
L being C₁₋₅₀ alkyl or C₁₋₅₀ heteroalkyl; 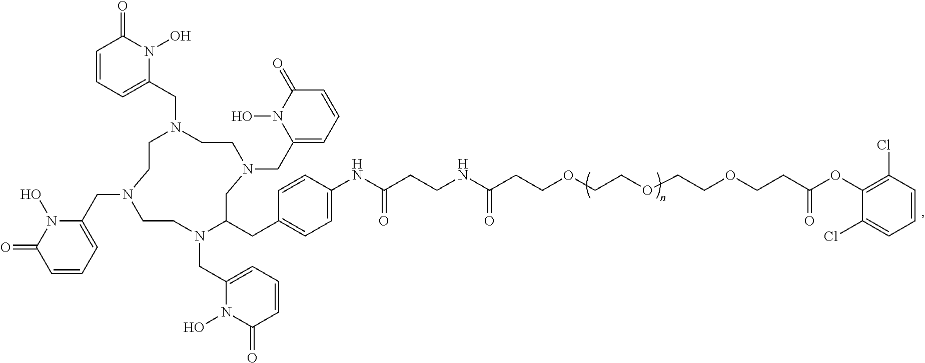
n being an integer of 1-10; and 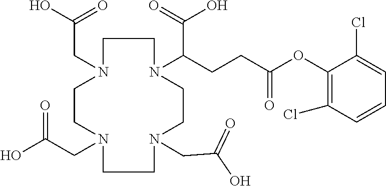 [SMILES: O=C(O)CN1CCN(CC(=O)O)CCN(C(CCC(=O)Oc2c(Cl)cccc2Cl)C(=O)O)CCN(CC(=O)O)CC1]
or a stereoisomer thereof.

16. The method of claim 15, wherein the compound of A-L-C(═O)-M-X has the following structure:

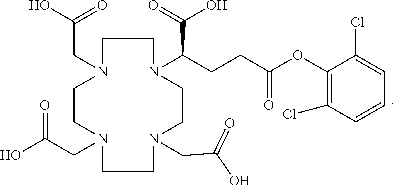 [SMILES: O=C(O)CN1CCN(CC(=O)O)CCN([C@H](CCC(=O)Oc2c(Cl)cccc2Cl)C(=O)O)CCN(CC(=O)O)CC1]

17. The method of claim 1, wherein the compound of A-L-C(═O)-M-X is selected from the group consisting of 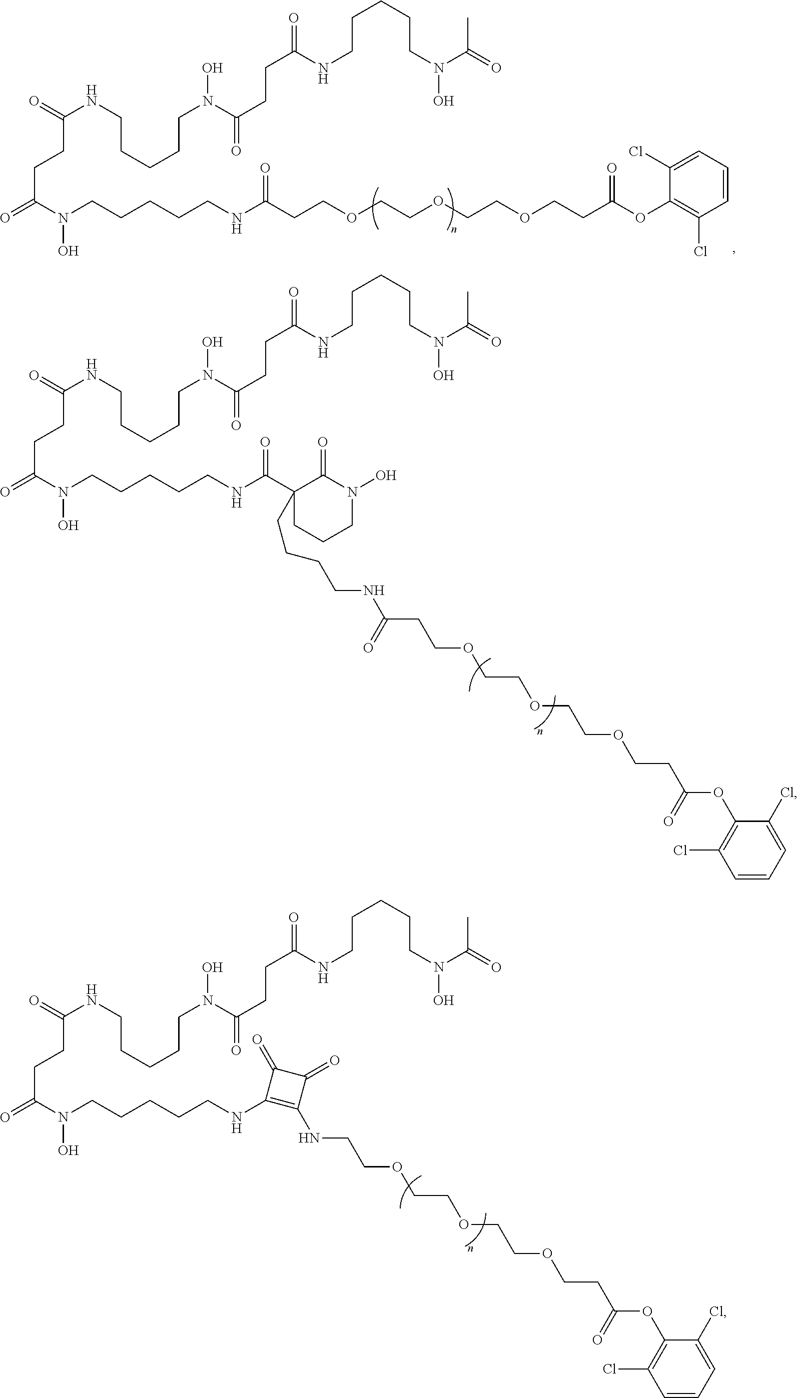 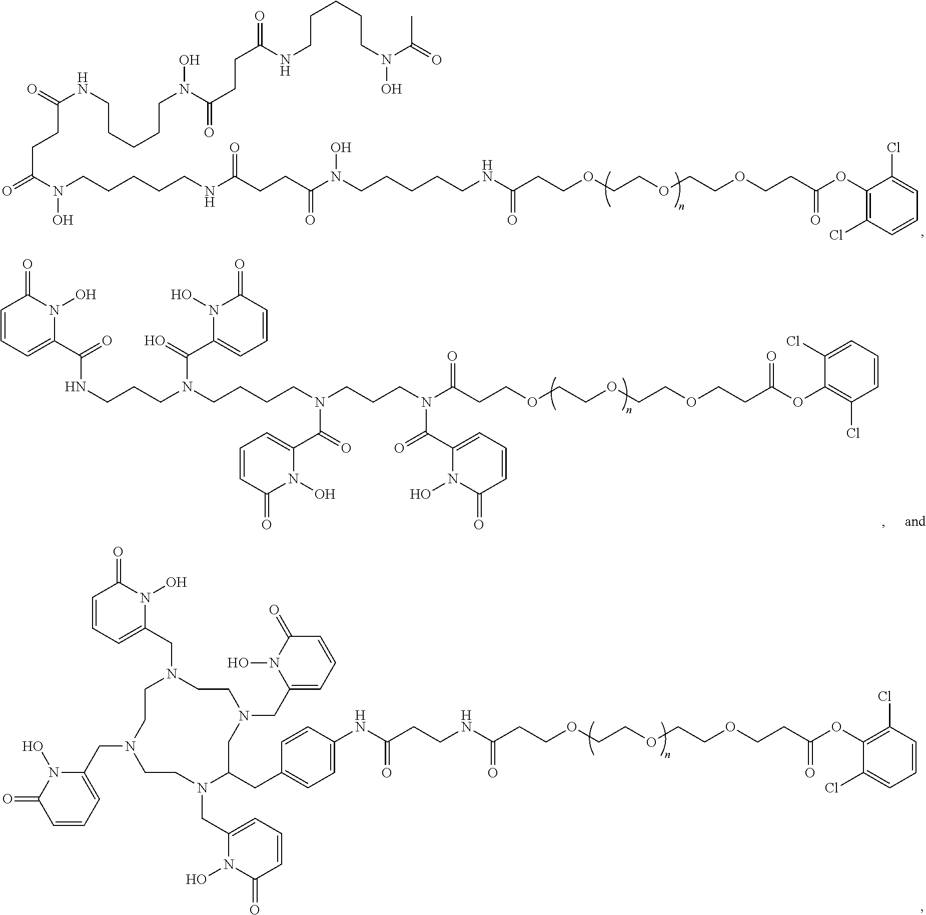
wherein each n, independently, is an integer of 1-10.

18. A compound having the structure of formula (II) below, or a metal complex thereof, or a salt thereof: 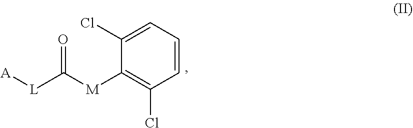
wherein
A is C₁₋₅₀ heteroalkyl comprising one or more hydroxamic acid units, C₁₋₅₀ heteroalkyl substituted directly or indirectly with one or more hydroxypyridinone units, or 5-20 membered heterocycloalkyl substituted directly or indirectly with one or more acetic acid or hydroxypyridinone units;
L is a linker selected from the group consisting of C₁₋₅₀ alkyl, C₁₋₅₀ heteroalkyl, C3-20 cycloalkyl, C₄₋₂₀ cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted; and
M is Oor S.

19. The compound of claim 18, wherein A is C₁₋₅₀ heteroalkyl comprising one or more hydroxamic acid units, wherein the heteroalkyl group is optionally substituted with one or more oxo (═O).

20. The compound of claim 19, wherein A has the structure shown below:

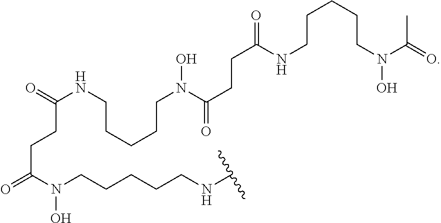 [SMILES: *NCCCCCN(O)C(=O)CCC(=O)NCCCCCN(O)C(=O)CCC(=O)NCCCCCN(O)C(C)=O]

21. The compound of claim 18, wherein A is C₁₋₅₀ heteroalkyl substituted directly or indirectly with one or more hydroxypyridinone units, wherein the heteroalkyl group comprises two or more nitrogen atoms.

22. The compound of claim 21, wherein A has the structure shown below:

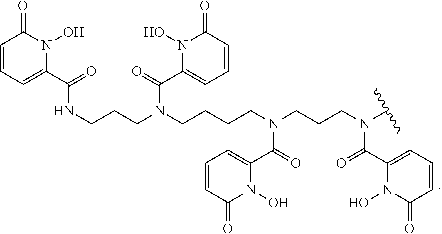 [SMILES: *N(CCCN(CCCCN(CCCNC(=O)c1cccc(=O)n1O)C(=O)c1cccc(=O)n1O)C(=O)c1cccc(=O)n1O)C(=O)c1cccc(=O)n1O]

23. The compound of claim 18, wherein A is 5-20 membered heterocycloalkyl substituted directly or indirectly with one or more hydroxypyridinone units, wherein the 5-20 membered heterocycloalkyl group comprises three or more nitrogen atoms.

24. The compound of claim 23, wherein A has the structure shown below:

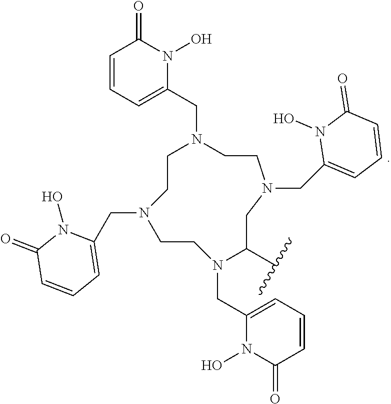 [SMILES: *C1CN(Cc2cccc(=O)n2O)CCN(Cc2cccc(=O)n2O)CCN(Cc2cccc(=O)n2O)CCN1Cc1cccc(=O)n1O]

25. The compound of any one of claims 18-24, wherein L is optionally substituted C₁₋₅₀ alkyl, optionally substituted C₁₋₅₀ heteroalkyl, C═O, —NH—(C═O)—, or a combination thereof, wherein the optionally substituted C1-50 heteroalkyl comprises one or more oxygen atoms.

26. The compound of claim 25, wherein L comprises 3-20 polyethylene glycol (PEG) units.

27. The compound of claim 18, selected from one of the following: 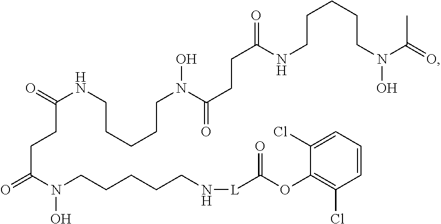
in which L is a linker selected from the group consisting of C₁₋₅₀ alkyl, C₁₋₅₀ heteroalkyl, C₃₋₂₀ cycloalkyl, C₄₋₅₀ cycloalkenyl, C₃₋₂₀ heterocycloalkyl, aryl, heteroaryl, C═O, —NR—(C═O)—, and a combination thereof, R being H or C₁₋₆ alkyl, and each of alkyl, heteroalkyl, cycloalkyl, cycloalkenyl, heterocycloalkyl, aryl, and heteroaryl being optionally substituted; 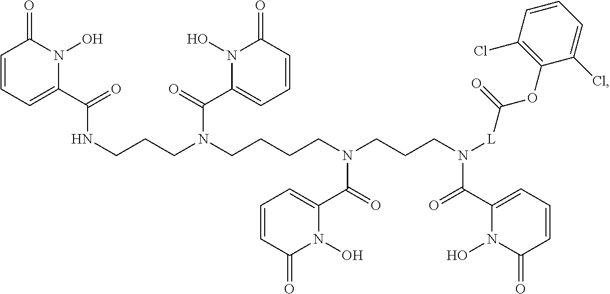
L being C₁₋₅₀ alkyl or C₁₋₅₀ heteroalkyl; 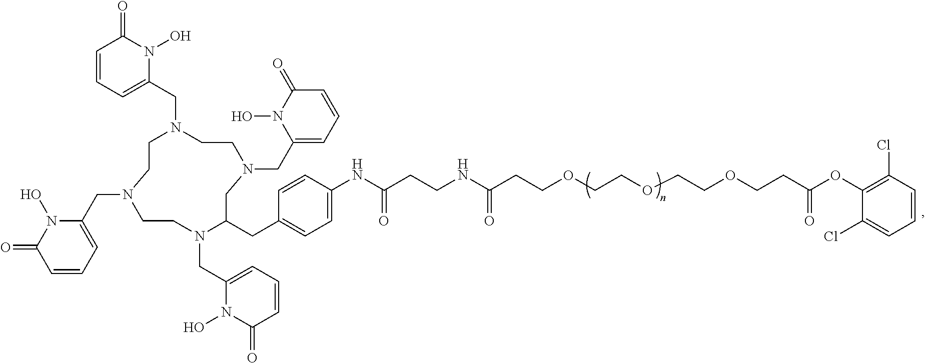
n being an integer of 1-10; and 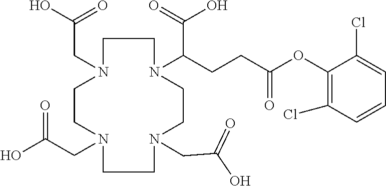 [SMILES: O=C(O)CN1CCN(CC(=O)O)CCN(C(CCC(=O)Oc2c(Cl)cccc2Cl)C(=O)O)CCN(CC(=O)O)CC1]
or a stereoisomer thereof.

28. The compound of claim 27, having the following structure:

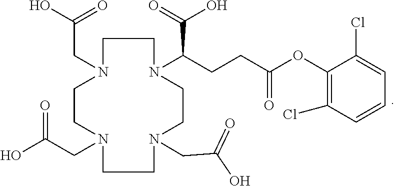 [SMILES: O=C(O)CN1CCN(CC(=O)O)CCN([C@H](CCC(=O)Oc2c(Cl)cccc2Cl)C(=O)O)CCN(CC(=O)O)CC1]

29. The compound of claim 18, selected from one of the following: 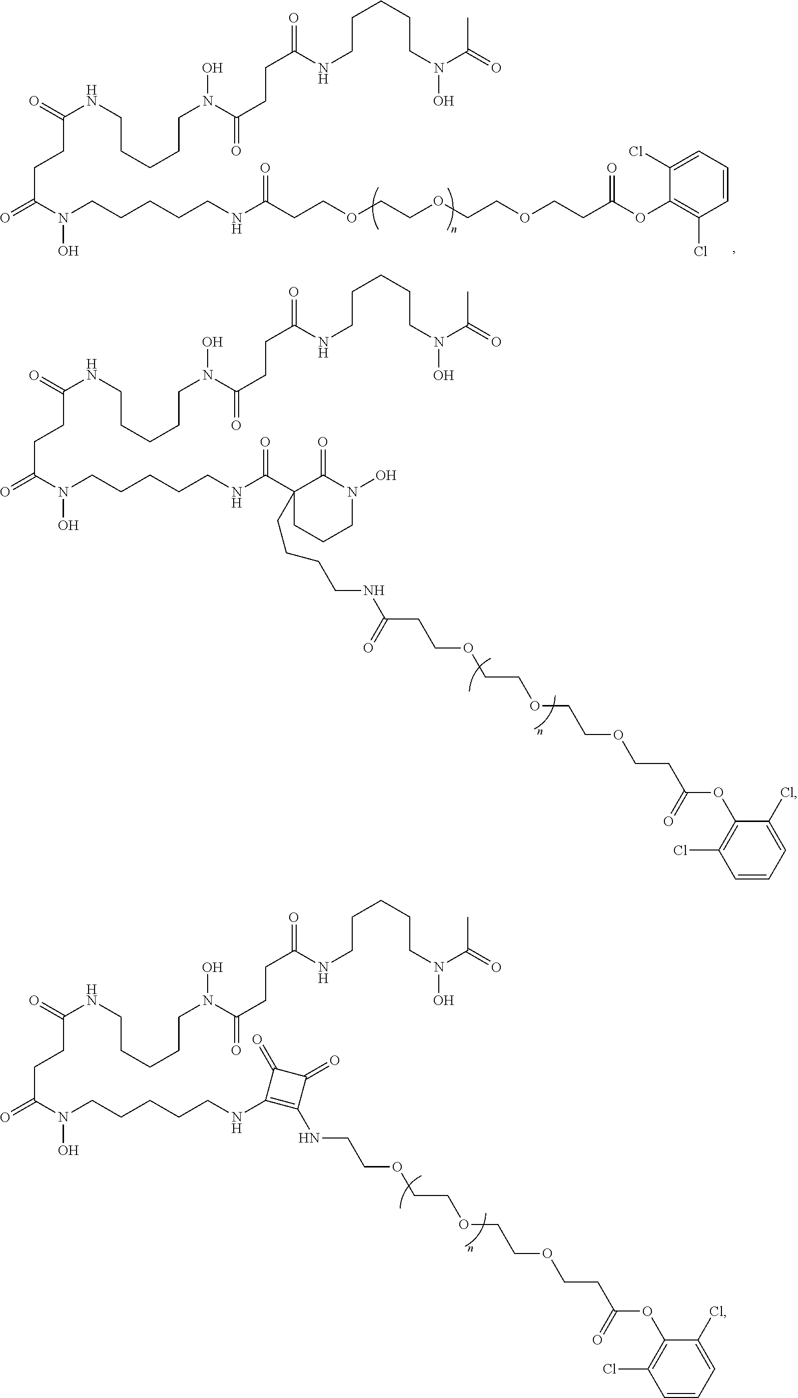 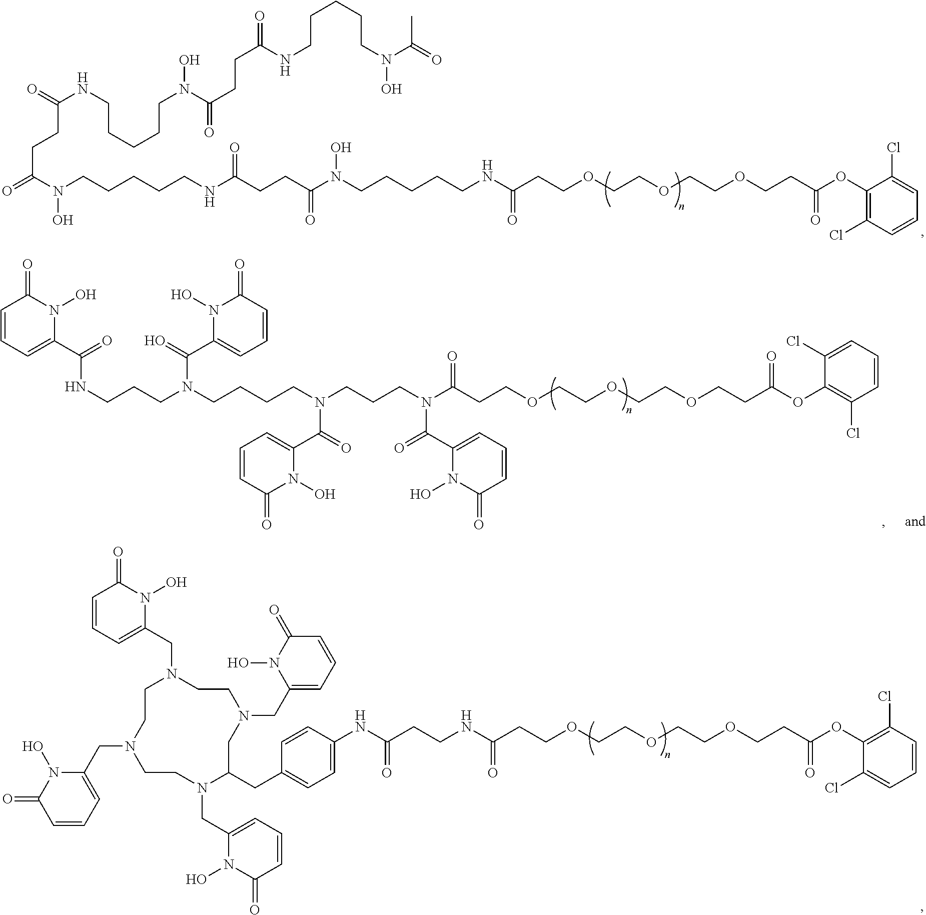
wherein each n, independently, is an integer of 1-10.

30. The compound of any one of claims 18-29, wherein the metal of said metal complex is selected from the group consisting of Bi, Pb, Y, Mn, Cr, Fe, Co, Zn, Ni, Tc, In, Ga, Cu, Re, a lanthanide, and an actinide; or the metal of said metal complex is a radionuclide selected from the group consisting of ⁸⁹Zr, ⁴⁷Sc, ⁵⁵Co, ⁶⁰Cu, ⁶¹Cu, C⁶²Cu, ⁶⁴Cu, ⁶⁷Cu, ⁶⁶Ga, ⁶⁷Ga, ⁶⁸Ga, ⁸²Rb, ⁸⁶Y, ⁸⁷Y, ⁹⁰Y, ⁹⁷Ru, ¹⁰⁵Rh, ¹⁰⁹Pd, ¹¹¹In, ^{117m}Sn, ¹⁴⁹Pm, ⁵²Mn, ¹⁴⁹Tb, ¹⁵²Tb, ¹⁵³Sm, ¹⁷⁷Lu, ¹⁸⁶Re, ¹⁸⁸Re, ¹⁹⁹Au, ²⁰¹Tl, ²⁰³Pb, ²¹²Pb, ²¹²Bi, ²¹³Bi, ²²⁵Ac, ²²⁷Ac, ²²³Ra and ²²⁷Th.
